(19) United States
(12) Patent Application Publication
(10) Pub. No.: US 2024/0350506 A1
(43) Pub. Date: Oct. 24, 2024

(54) PREPARATION OF HISTONE DEACETYLASE INHIBITOR, AND PREPARATION METHOD THEREFOR AND USE THEREOF

(71) Applicant: CHENGDU ZENITAR BIOMEDICAL TECHNOLOGY CO., LTD., Chengdu, Sichuan (CN)

(72) Inventor: Li Juan CHEN, Chengdu, Sichuan (CN)

(21) Appl. No.: 18/579,906

(22) PCT Filed: Jul. 18, 2022
(86) PCT No.: PCT/CN2022/106270

§ 371 Date: Jan. 17, 2024

(30) Foreign Application Priority Data
Jul. 19, 2021 (CN) 202110815779.2

(51) Int. Cl. A61K 31/5377; A61K 31/52; A61K 47/18; A61K 47/26; A61K 47/40

(52) U.S. Cl. CPC A61K 31/5377; A61K 31/52; A61K 47/183; A61K 47/26; A61K 47/40

(57) ABSTRACT

A preparation of a histone deacetylase inhibitor contains the histone deacetylase inhibitor and an excipient. The excipient is at least one of or a combination of two or more of cyclodextrin, arginine and meglumine. The preparation significantly increases the solubility of the histone deacetylase inhibitor in water, and improves the stability and retains the excellent anti-tumor activity of the histone deacetylase inhibitor.

### FIELD OF THE INVENTION

The present invention belongs to the field of biomedicine, and specifically relates to a preparation of a histone deacetylase inhibitor (HDACI), as well as a preparation method therefor and uses thereof.

### BACKGROUND OF THE INVENTION

Histones are positively charged small proteins, and are rich in alkaline amino acids (positively charged under physiological conditions). These amino acids interact with the phosphate groups (negatively charged under physiological pH conditions) of DNA. There mainly are five types of histones: H1, H2A, H2B, H3, and H4. The amino acid sequences of histones H2A, H2B, H3, and H4 show significant conservation among species. Most histones are synthesized in the S phase of the cell cycle, and newly synthesized histones quickly enter the nucleus and connect with DNA. Within a few minutes of its synthesis, the new DNA connects with histones in the nucleosome structure.

A small portion of the histone structure, especially their amino acid side chains, can be modified by enzymes, that is performed by adding methyl, acetyl, or phosphate groups to neutralize the positive charge of the side chains or convert them into negative charges after translation. For example, lysine and arginine groups can be methylated, lysine groups can be acetylated, and serine groups can be phosphorylated. As for lysine, the side chain —(CH₂)—NH₂ can be acetylated by enzymes such as acetyltransferase, to provide amide —(CH₂)₄—NHC(═O)CH₃. Methylation, acetylation, and phosphorylation of the N-terminal of histones extending from the center of nucleosomes can affect the chromatin structure and gene expression.

The acetylation and deacetylation of histones are related to transcriptional events that lead to cell proliferation and/or differentiation. With further research on histones, it has been found that histone deacetylases (HDACs) can regulate gene transcription and chromatin remodeling by catalyzing the deacetylation of histone N-terminal lysine residues. In addition, HDACs can also catalyze the deacetylation of proteins other than histones, such as p21, tubulin, HSP90 (Heat shock protein 90), etc.

HDACs are closely related to the occurrence of various diseases, and it has been found that HDACs are implicated in the occurrence of tumors. Inhibiting HDACs can induce the arrest, differentiation, and apoptosis of tumor cell cycle.

However, many potential inhibitors have at least the following issues: (1) the solubility in water is low, and too high or low pH values are often required to make inhibitors dissolve; (2) there is physical and/or chemical instability in aqueous solutions.

Therefore, providing formulations or prodrugs comprising HDACIs that can address the aforementioned issues will enable effective dissolution of HDACIs in water, while improve their stability and retain their biological activity. This will greatly extend the application of HDACIs, and thus have significant clinical significance and value.

### SUMMARY OF THE INVENTION

The object of the present invention is to provide a preparation of an HDACI, as well as a preparation method therefor and uses thereof.

The present invention provides a pharmaceutical composition, which comprises an HDACI and an excipient;

The HDACI is a compound disclosed in the Chinese patent publication number CN107849045B, or a pharmaceutically acceptable salt, a solvate, an amide, an ester, an ether, a chemically protected form, and a prodrug thereof.

The excipient is at least one of or a combination comprising two or more of cyclodextrin, arginine, and meglumine.

Further, the HDACI is a compound represented by formula I or a pharmaceutically acceptable salt, a solvate, an amide, an ester, an ether, a chemically protected form, and a prodrug thereof:

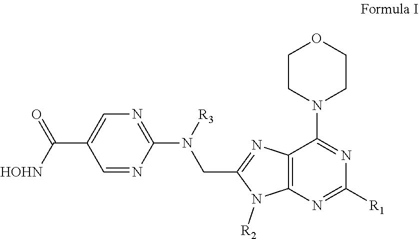

wherein, R₁ is selected from the group consisting of C₁-C₄ alkyl, C₁-C₄ alkoxy, —OH, halogen, C₃-C₈ cycloalkyl, —NH₂,

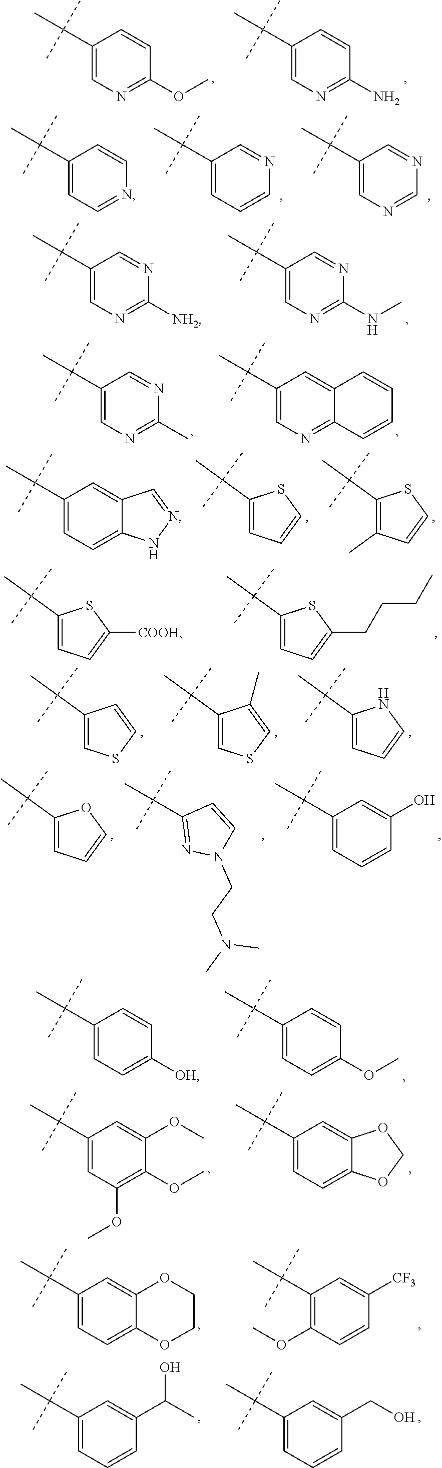

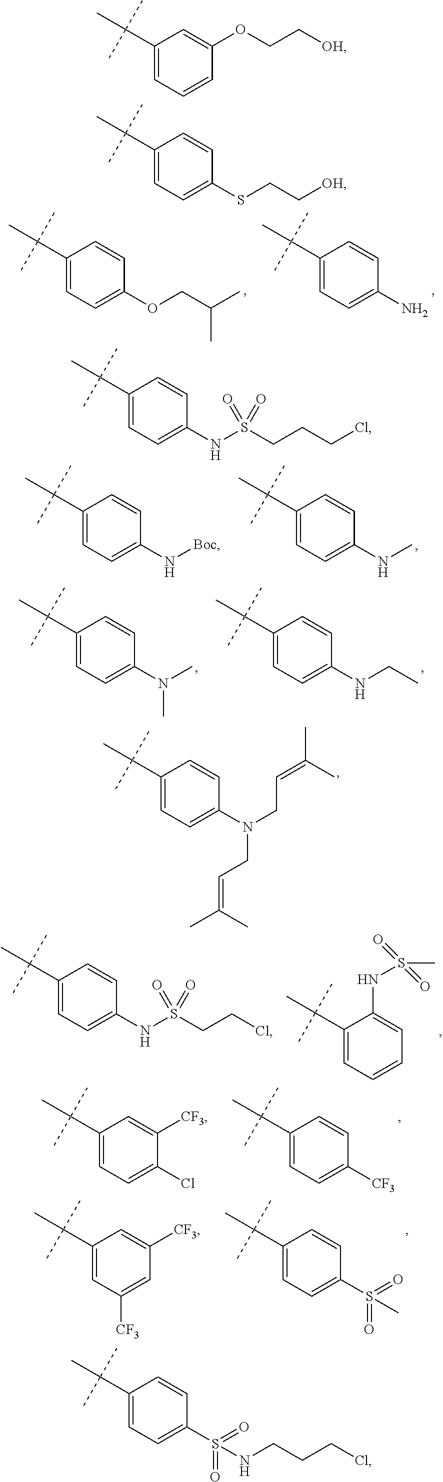

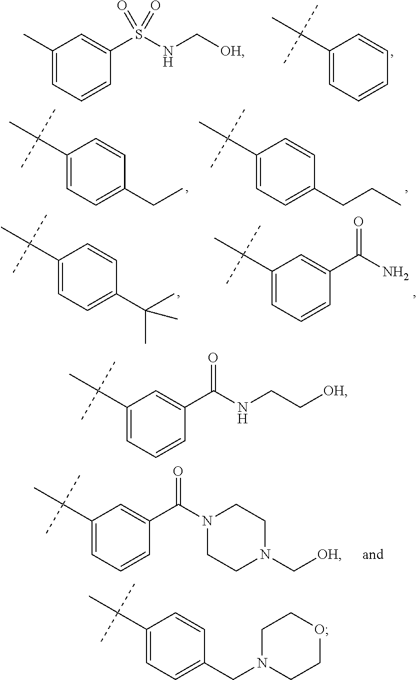

R₂ is selected from the group consisting of C₁-C₄ alkoxy or C₃-C₈ cycloalkyl;
R₃ is selected from the group consisting of —H, C₁-C₄ alkyl, C₁-C₄ alkoxy, —OH, halogen or C₃-C₈ cycloalkyl;
the mass ratio of cyclodextrin to the inhibitor is (10-20):1; and/or the mass ratio of arginine to the inhibitor is (2-4):1; and/or the mass ratio of meglumine to the inhibitor is (1.5-6):1.

The pharmaceutical composition mentioned above, wherein the compound represented by formula I is selected from the group consisting of:

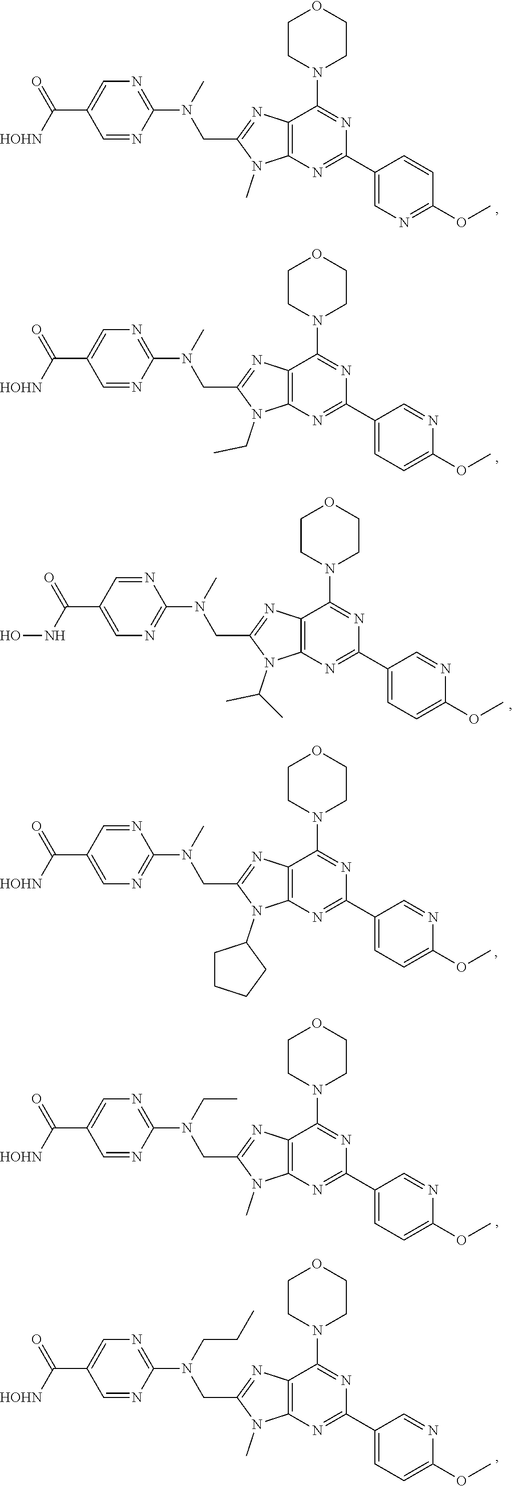

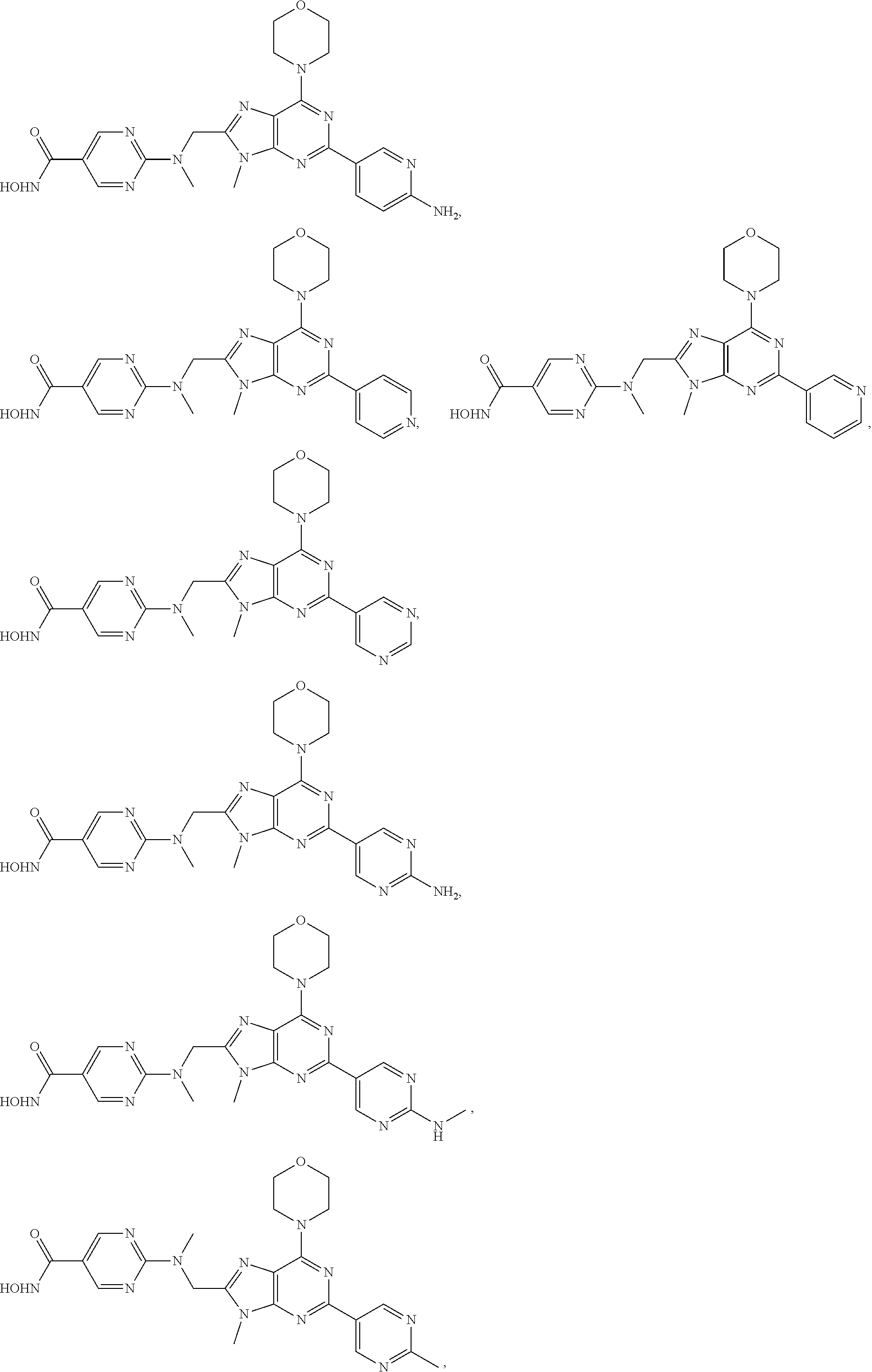

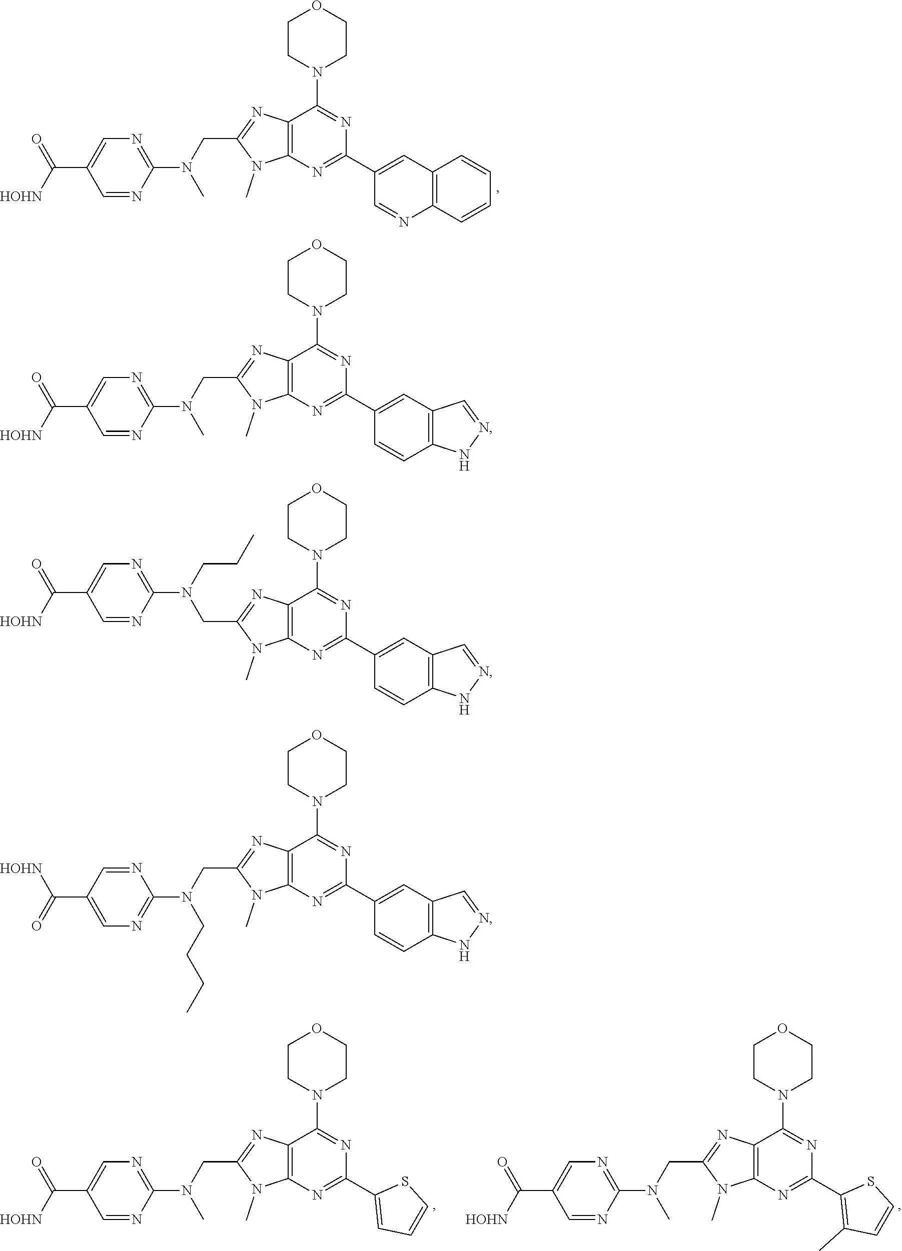

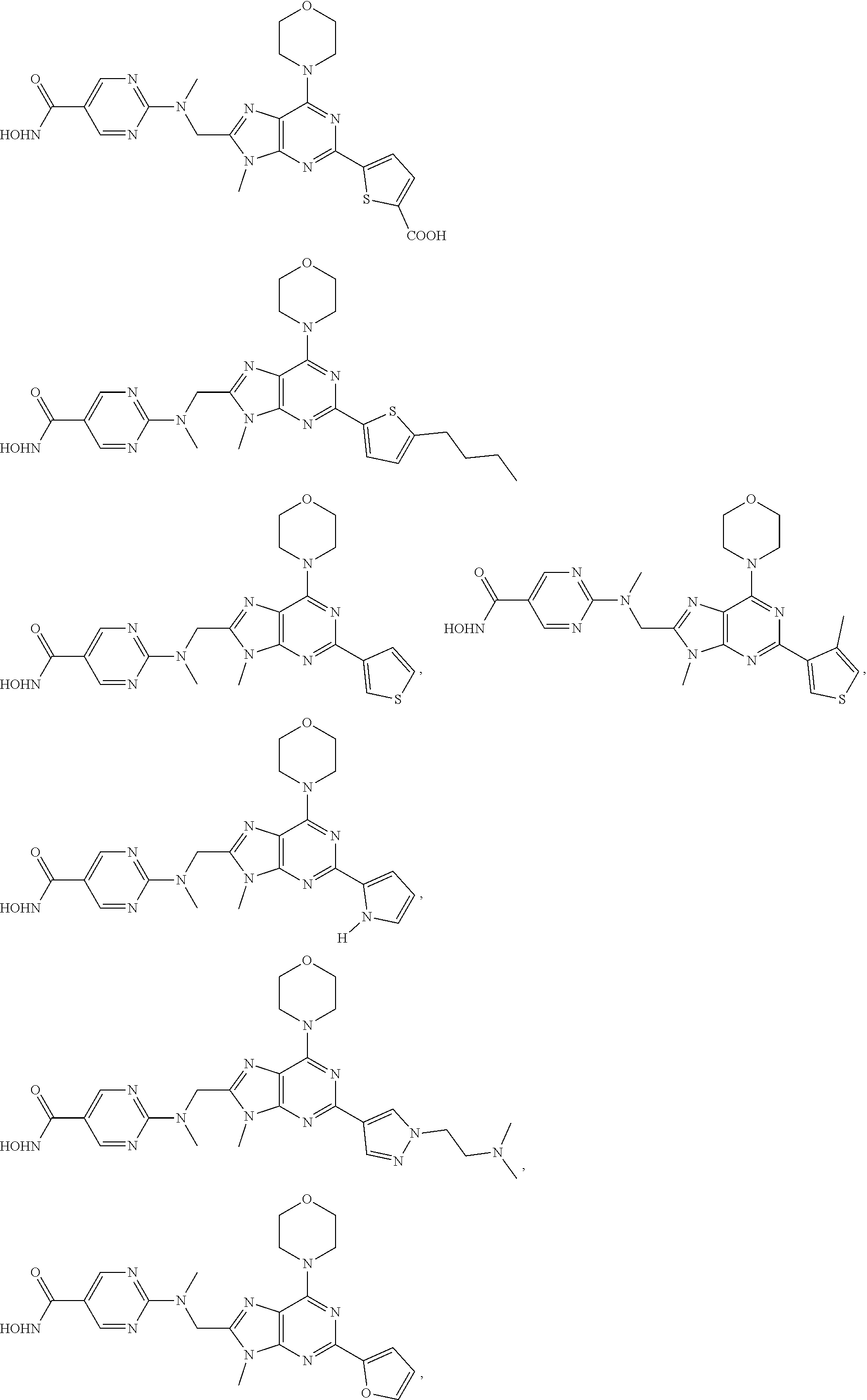

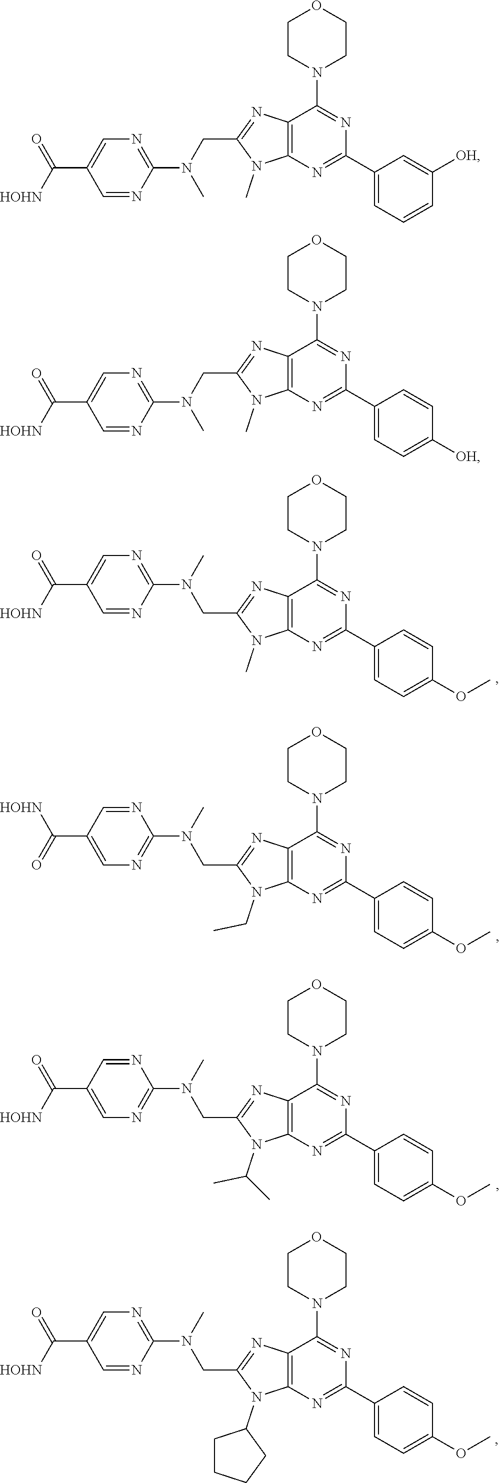

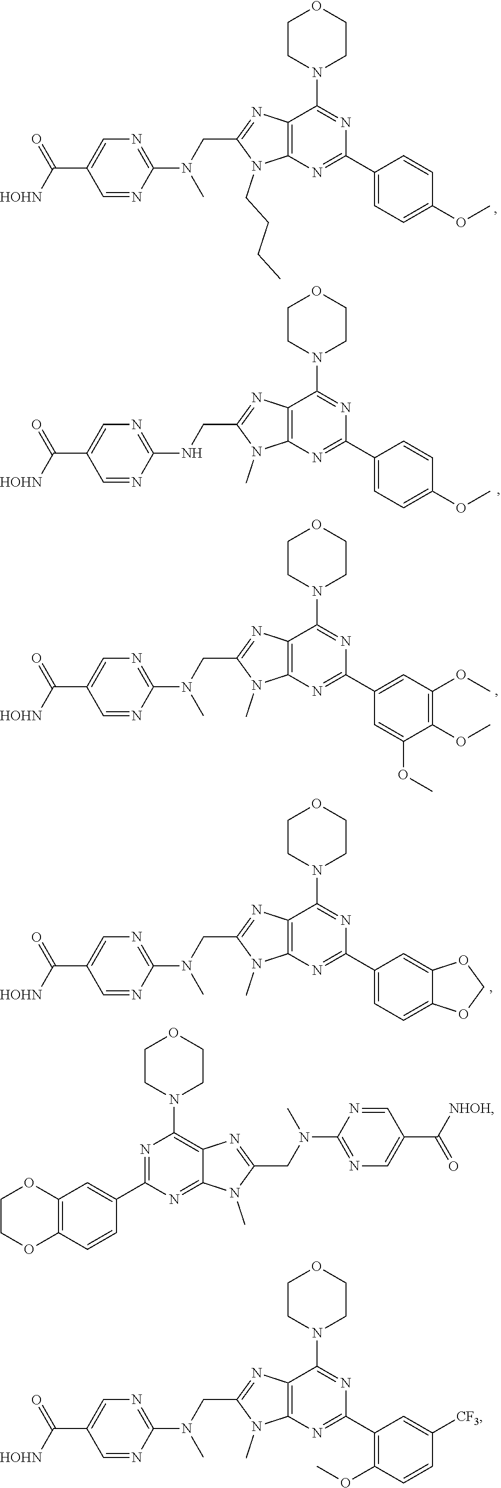

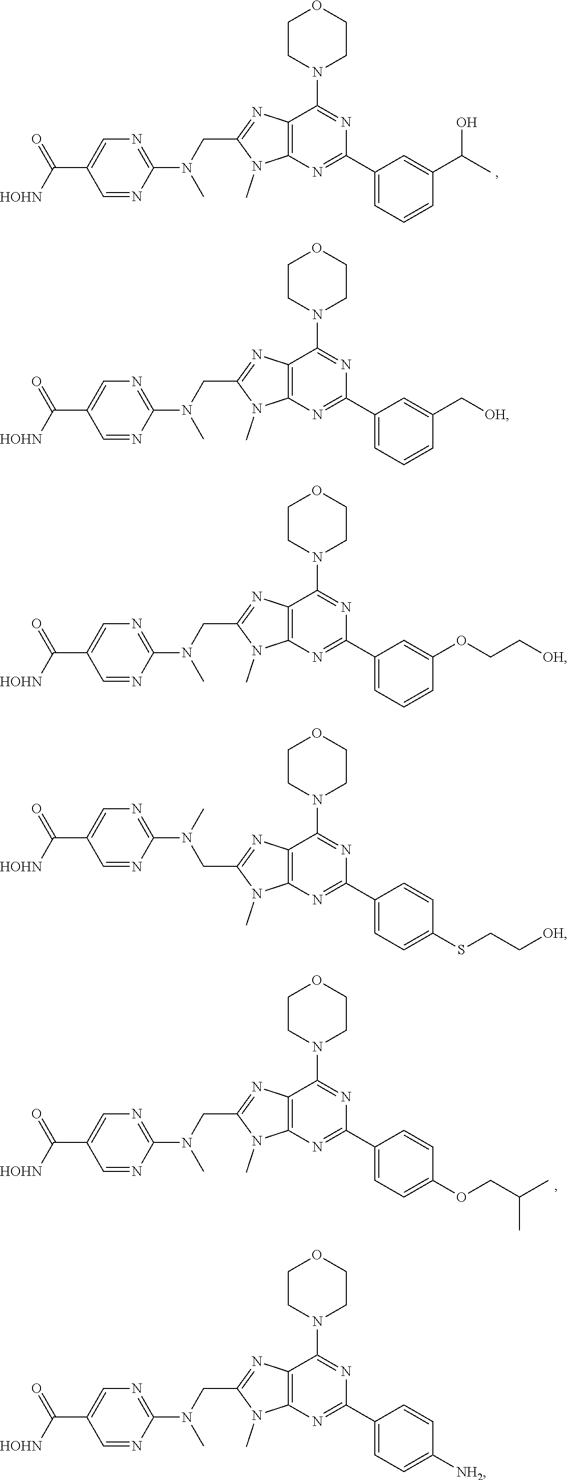

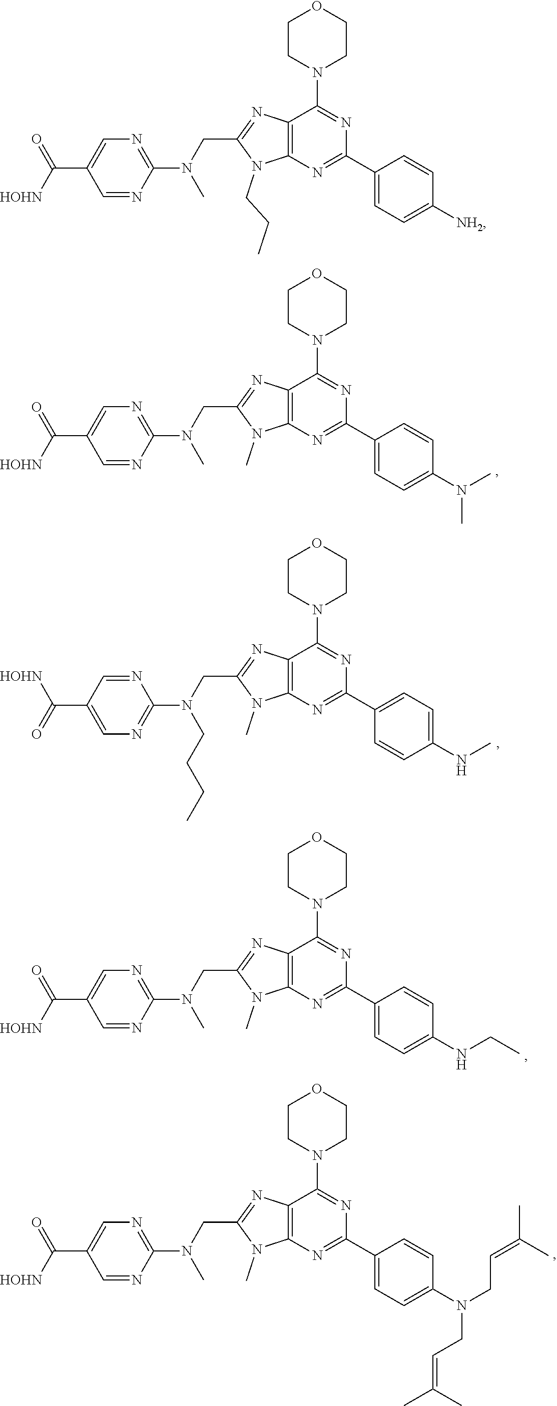

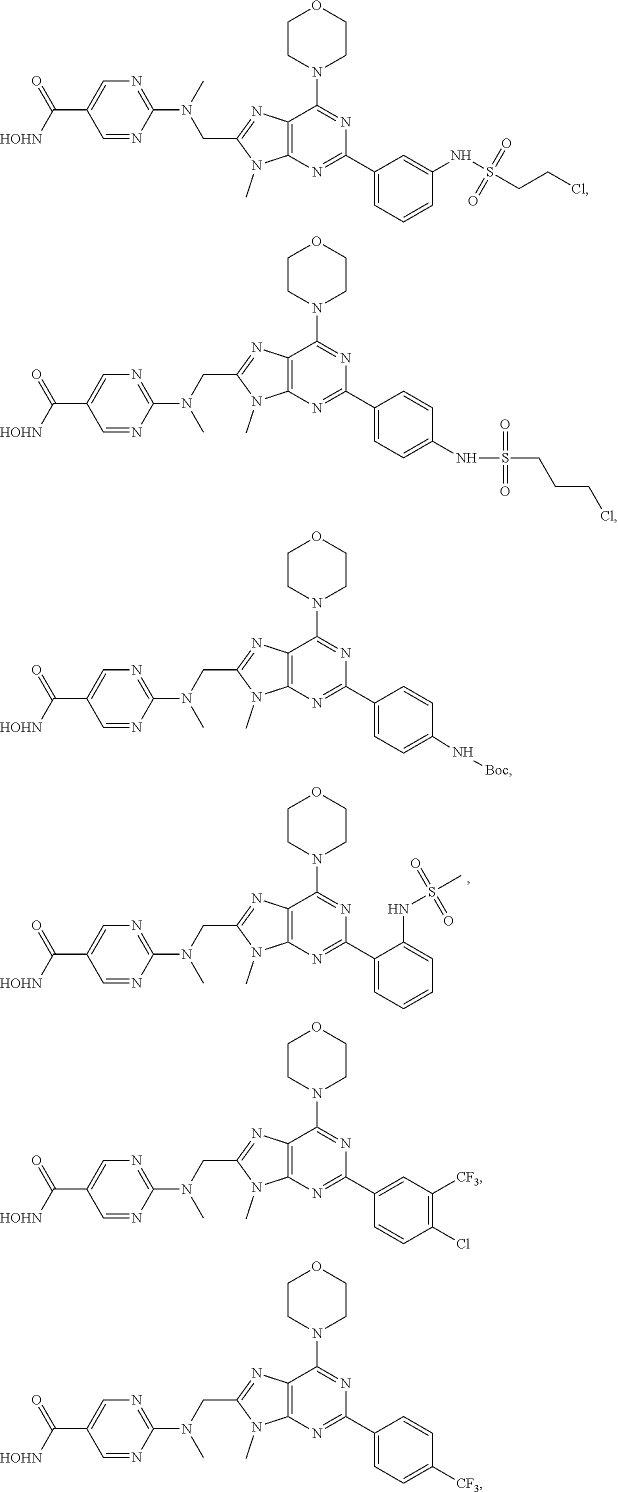

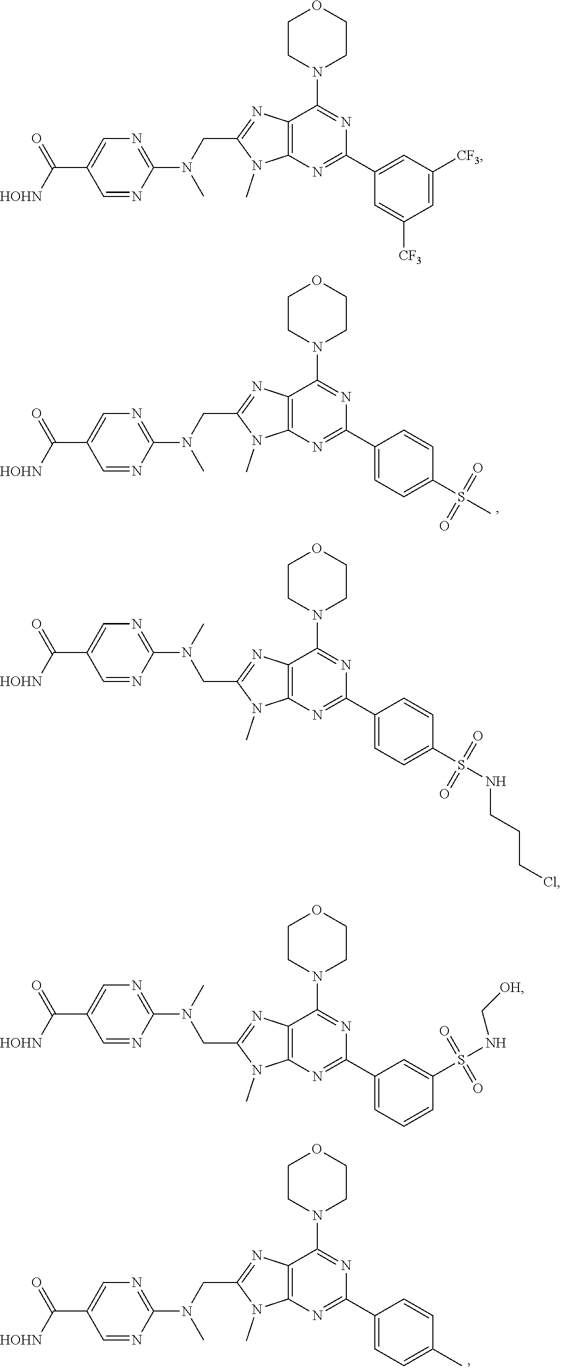

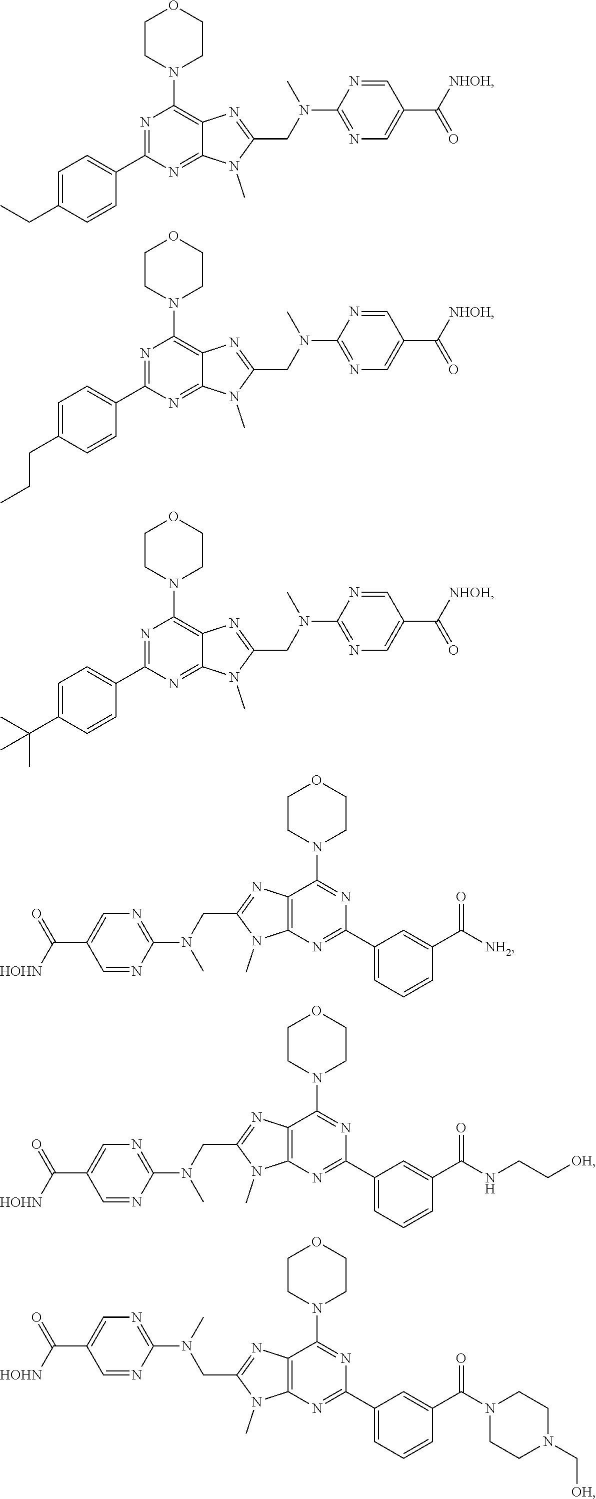

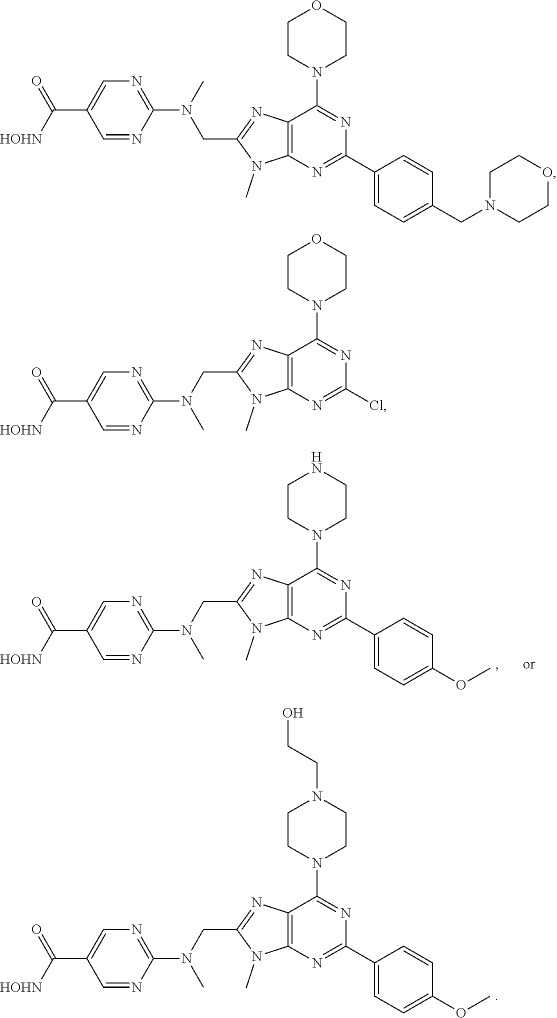

Preferably, the above excipient is selected from the group consisting of cyclodextrin, arginine, and meglumine, and the mass ratio of cyclodextrin to the inhibitor is (10-20):1, and preferably (15-20):1; the mass ratio of arginine to the inhibitor is (2-4):1; the mass ratio of meglumine to the inhibitor is (1.5-6):1;

More preferably, the above excipient is selected from the group consisting of cyclodextrin, arginine, and meglumine, and the mass ratio of cyclodextrin, arginine, meglumine and the inhibitor is 15:4:1.5:1.

Further, said cyclodextrin is selected from the group consisting of α-cyclodextrin, β-cyclodextrin, γ-cyclodextrin, (C1-4 alkyl)-α-cyclodextrin, (C1-4 alkyl)-β-cyclodextrin, (C1-4 alkyl)-γ-cyclodextrin, hydroxyl-(C1-4 alkyl)-α-cyclodextrin, hydroxyl-(C1-4 alkyl)-β-cyclodextrin, hydroxyl-(C1-4 alkyl)-γ-cyclodextrin, carboxyl-(C1-4 alkyl)-α-cyclodextrin, carboxyl-(C1-4 alkyl)-β-cyclodextrin, carboxyl-(C1-4 alkyl)-γ-cyclodextrin, α-cyclodextrin ethers, β-cyclodextrin ethers, γ-cyclodextrin ethers, α-cyclodextrin sulfobutyl ether, β-cyclodextrin sulfobutyl ether, and γ-cyclodextrin sulfobutyl ether; preferably, said cyclodextrin is hydroxypropyl-β-cyclodextrin.

Further, the above pharmaceutical composition is a preparation formed by the deacetylase inhibitor and excipients, in combination with pharmaceutically acceptable auxiliary ingredients.

More further, the preparation mentioned above is an injection, and the pharmaceutically acceptable auxiliary ingredients are water for injection, saline, glucose aqueous solution, saline for injection and infusion, glucose solution for injection and infusion, Ringer's solution, or Ringer's solution containing lactate; preferably, the pharmaceutically acceptable auxiliary ingredients are saline or glucose aqueous solution.

More further, the concentration of HDACIs in the liquid formulation is 0.1-1000 mg/mL; preferably, the concentration of HDACIs is 200-500 mg/mL; and more preferably, the concentration of HDACIs is 500 mg/mL.

The present invention also provides the preparation method of the above preparation, which comprises the following steps:
(1) The excipients are dissolved in the pharmaceutically acceptable auxiliary ingredients in a pre-determined ratio, to obtain the excipient solution;
(2) The HDACI is added to the excipient solution obtained in step (1), and then the resultant solution is stirred to dissolve, and filtered to obtain the preparation;
preferably, the temperature for stirring and dissolving is 30-50° C., and the time is 0.5-2 h;
more preferably, the temperature for stirring and dissolving is 50° C., and the time is 2 h

The experimental results have indicated that the preparation of the present invention significantly increases the solubility of HDACIs in water, improves the stability of HDACIs, and retains the excellent anti-tumor activity of HDACIs, suggesting important values in clinical practice.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Phase solubility curves of purinostat mesylate in HP-β-CD and SBE-β-CD.

FIG. 2. Phase solubility curves of purinostat mesylate in arginine and meglumine.

FIG. 3. Molecular dynamics simulation diagram of hydroxypropyl-β-cyclodextrin including a medicament.

FIG. 4. Tumor images after treating Raji tumor model with the preparation of the present invention.

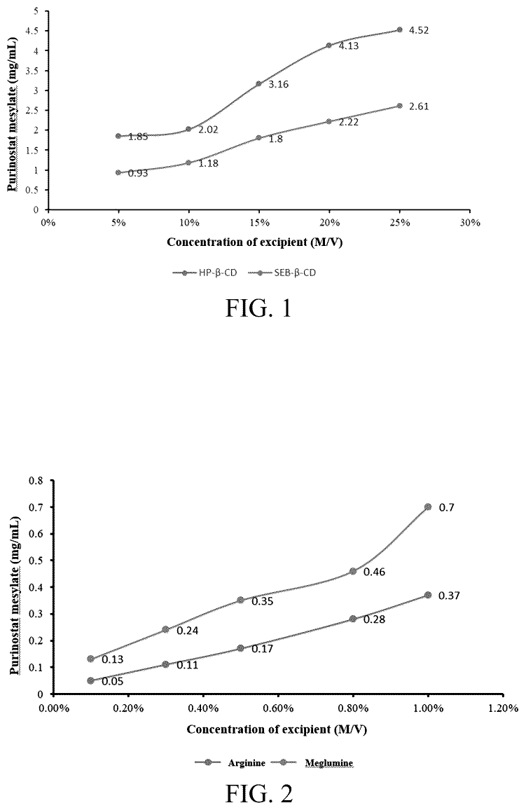

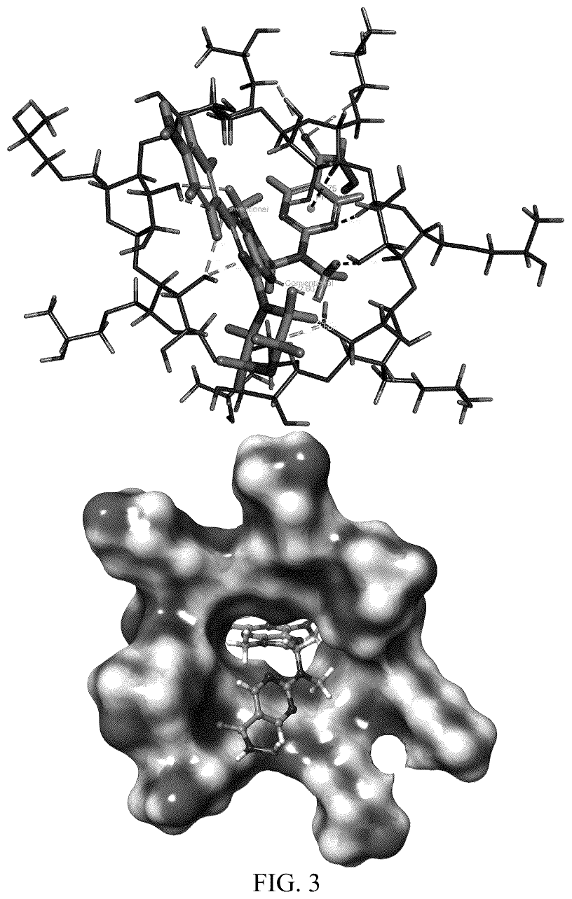

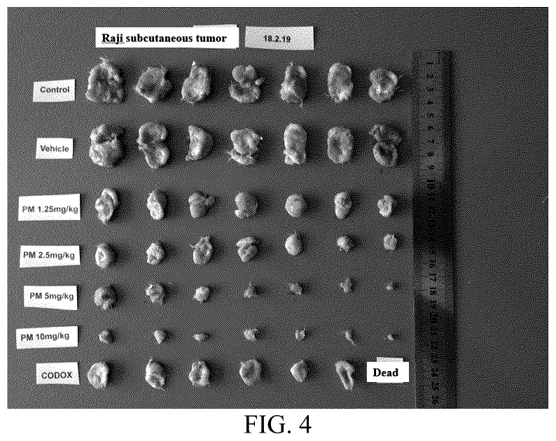

### EXAMPLES

The HDACI used in the example of the present invention is a compound disclosed in the Chinese patent publication number CN107849045B, or a pharmaceutically acceptable salt, a solvate, an amide, an ester, an ether, a chemically protected form, and a prodrug thereof;

Other raw materials and equipment used in the present invention are known products obtained by purchasing those commercially available.

### Example 1. Preparation of a Formulation According to the Present Invention

The powder of an active pharmaceutical ingredient (API, 20 mg) was dissolved in 10% hydroxypropyl-β-cyclodextrin (HP-β-CD) solution by stirring in a water bath at 50° C. for 2 h. Then, the solution was filtered using a 0.22 m filter head to obtain an injectable solution containing 2 mg/mL of active substance, in which the mass ratio of HP-β-CD:API is 50:1.

The API is the mesylate of a compound with the following structure (i.e. purinostat mesylate):

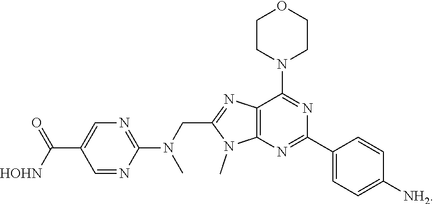

### Example 2. Preparation of a Formulation According to the Present Invention

The powder of an API (30 mg) was dissolved in a solution containing 10% HP-β-CD and 2% Arg by stirring in a water bath at 50° C. for 1 h. Then, the solution was filtered with a 0.22 μm filter head to obtain an injectable solution containing 3 mg/mL of active substance, in which the mass ratio of HP-β-CD:Arg:API is 100:20:3. The API is purinostat mesylate.

### Example 3. Preparation of a Formulation According to the Present Invention

The powder of an API (50 mg) was dissolved in a solution containing 10% HP-β-CD, 1% Arg and 1% meglumine by stirring in a water bath at 40° C. for 1 h. Then, the solution was filtered with a 0.22 μm filter head to obtain an injectable solution containing 5 mg/mL of active substance, in which the mass ratio of HP-β-CD:Arg:meglumine:API is 20:2:2:1. The API is purinostat mesylate.

### Example 4. Preparation of a Formulation According to the Present Invention

The powder of an API (30 mg) was dissolved in a solution containing 7.5% HP-β-CD and 2% Arg by stirring in a water bath at 50° C. for 2 h. Then, the solution was filtered with a 0.22 μm filter head to obtain an injectable solution containing 3 mg/mL of active substance, in which the mass ratio of HP-β-CD:Arg:API is 75:20:3. The API is purinostat mesylate.

### Example 5. Preparation of a Formulation According to the Present Invention

The powder of an API (50 mg) was dissolved in a solution containing 7.5% HP-β-CD, 2% Arg and 0.75% meglumine by stirring in a water bath at 50° C. for 2 h. Then, the solution was filtered with a 0.22 μm filter head to obtain an injectable solution containing 5 mg/mL of active substance, in which the mass ratio of HP-β-CD:Arg:meglumine:API is 15:4:1.5:1. The API is purinostat mesylate.

### Example 6. Preparation of a Formulation According to the Present Invention

The powder of an API (50 mg) was dissolved in a solution containing 5% HP-β-CD, 1% Arg and 1% meglumine by stirring in a water bath at 40° C. for 1 h. Then, the solution was filtered with a 0.22 μm filter head to obtain an injectable solution containing 5 mg/mL of active substance, in which the mass ratio of HP-β-CD:Arg:meglumine:API is 10:2:2:1. The API is purinostat mesylate.

### Example 7. Preparation of a Formulation According to the Present Invention

The powder of an API (50 mg) was dissolved in a solution containing 5% HP-β-CD, 2% Arg and 1% meglumine by stirring in a water bath at 40° C. for 1 h. Then, the solution was filtered with a 0.22 μm filter head to obtain an injectable solution containing 5 mg/mL of active substance, in which the mass ratio of IP-β-CD:Arg:meglumine:API is 10:4:2:1. The API is purinostat mesylate.

### Example 8. Preparation of a Formulation According to the Present Invention

The formulation was prepared by reference to the preparation method of Example 5. The API was the mesylate of

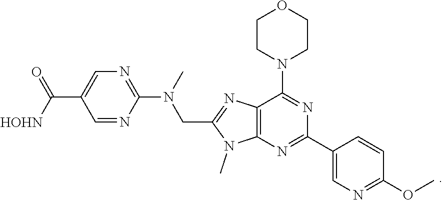

### Example 9. Preparation of a Formulation According to the Present Invention

The formulation was prepared by reference to the preparation method of Example 5. The API was the mesylate of

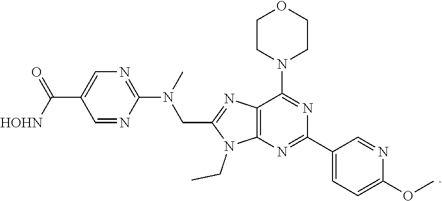

### Example 10. Preparation of a Formulation According to the Present Invention

The formulation was prepared by reference to the preparation method of Example 5. The API was the mesylate of

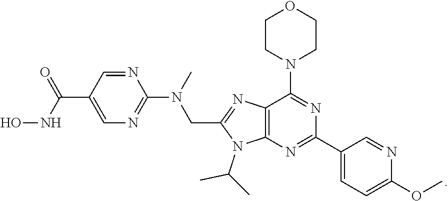 [SMILES: COc1ccc(-c2nc(N3CCOCC3)c3nc(CN(C)c4ncc(C(=O)NO)cn4)n(C(C)C)c3n2)cn1]

### Example 11. Preparation of a Formulation According to the Present Invention

The formulation was prepared by reference to the preparation method of Example 5. The API was the mesylate of

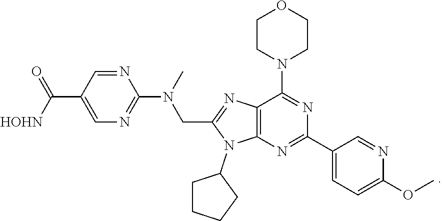

### Example 12. Preparation of a Formulation According to the Present Invention

The formulation was prepared by reference to the preparation method of Example 5. The API was the mesylate of

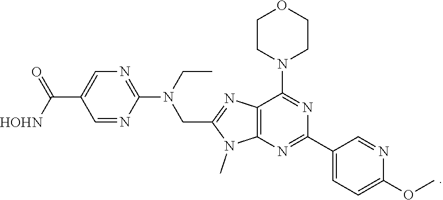

### Example 13. Preparation of a Formulation According to the Present Invention

The formulation was prepared by reference to the preparation method of Example 5. The API was the mesylate of

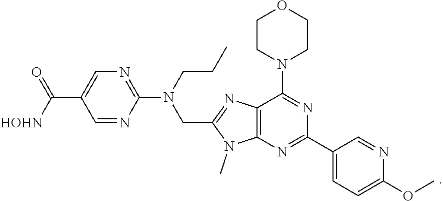

### Example 14. Preparation of a Formulation According to the Present Invention

The formulation was prepared by reference to the preparation method of Example 5. The API was the mesylate of

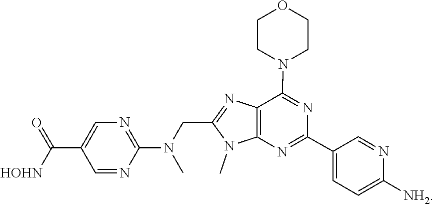

### Example 15. Preparation of a Formulation According to the Present Invention

The formulation was prepared by reference to the preparation method of Example 5. The API was the mesylate of

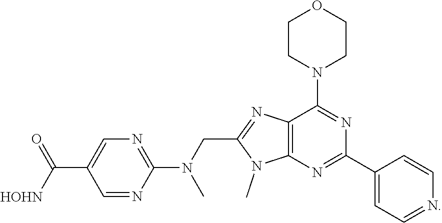

### Example 16. Preparation of a Formulation According to the Present Invention

The formulation was prepared by reference to the preparation method of Example 5. The API was the mesylate of

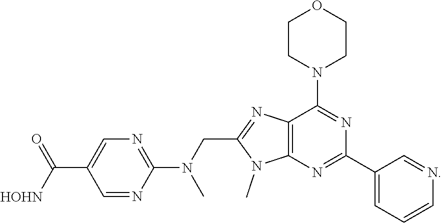

### Example 17. Preparation of a Formulation According to the Present Invention

The formulation was prepared by reference to the preparation method of Example 5. The API was the mesylate of

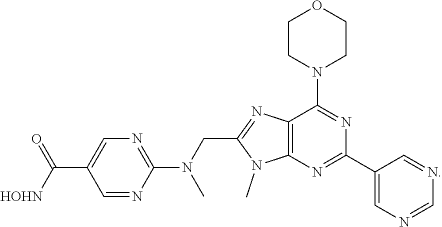

### Example 18. Preparation of a Formulation According to the Present Invention

The formulation was prepared by reference to the preparation method of Example 5. The API was the mesylate of

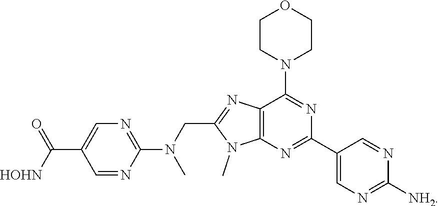

### Example 19. Preparation of a Formulation According to the Present Invention

The formulation was prepared by reference to the preparation method of Example 5. The API was the mesylate of

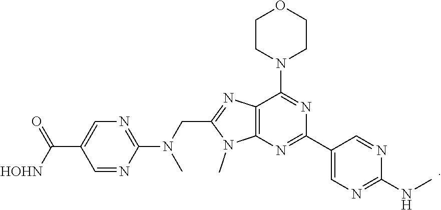

### Example 20. Preparation of a Formulation According to the Present Invention

The formulation was prepared by reference to the preparation method of Example 5. The API was the mesylate of

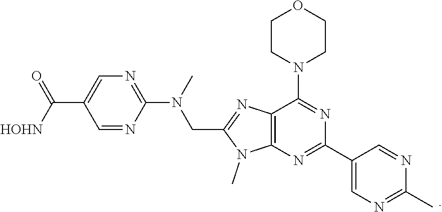

### Example 21. Preparation of a Formulation According to the Present Invention

The formulation was prepared by reference to the preparation method of Example 5. The API was the mesylate of

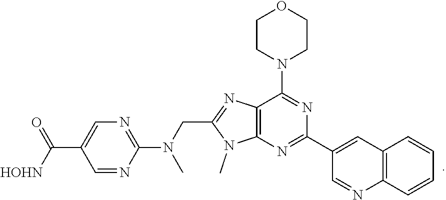

### Example 22. Preparation of a Formulation According to the Present Invention

The formulation was prepared by reference to the preparation method of Example 5. The API was the mesylate of

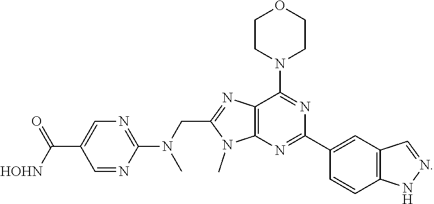

### Example 23. Preparation of a Formulation According to the Present Invention

The formulation was prepared by reference to the preparation method of Example 5. The API was the mesylate of

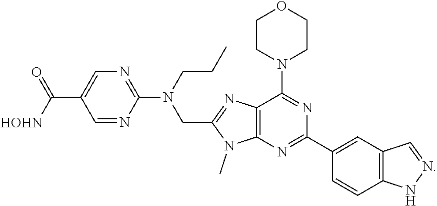

### Example 24. Preparation of a Formulation According to the Present Invention

The formulation was prepared by reference to the preparation method of Example 5. The API was the mesylate of

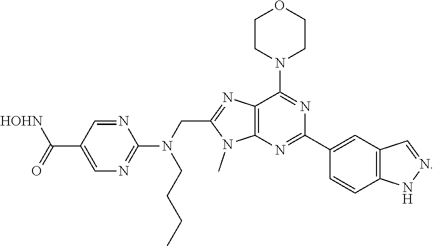

### Example 25. Preparation of a Formulation According to the Present Invention

The formulation was prepared by reference to the preparation method of Example 5. The API was the mesylate of

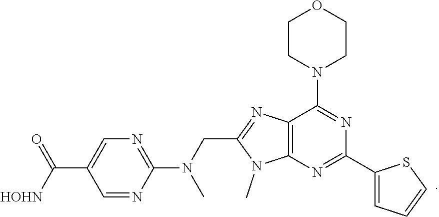

### Example 26. Preparation of a Formulation According to the Present Invention

The formulation was prepared by reference to the preparation method of Example 5. The API was the mesylate of

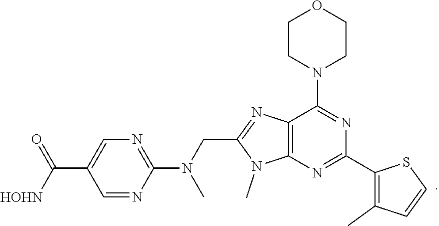

### Example 27. Preparation of a Formulation According to the Present Invention

The formulation was prepared by reference to the preparation method of Example 5. The API was the mesylate of

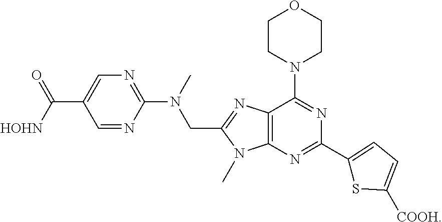

### Example 28. Preparation of a Formulation According to the Present Invention

The formulation was prepared by reference to the preparation method of Example 5. The API was the mesylate of

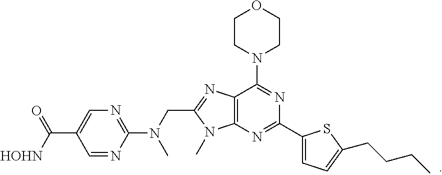

### Example 29. Preparation of a Formulation According to the Present Invention

The formulation was prepared by reference to the preparation method of Example 5. The API was the mesylate of

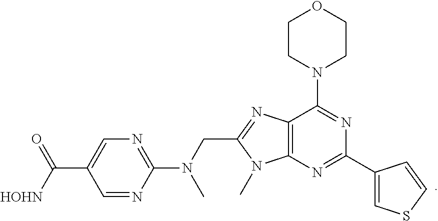

### Example 30. Preparation of a Formulation According to the Present Invention

The formulation was prepared by reference to the preparation method of Example 5. The API was the mesylate of

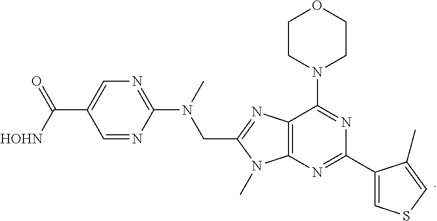

### Example 31. Preparation of a Formulation According to the Present Invention

The formulation was prepared by reference to the preparation method of Example 5. The API was the mesylate of

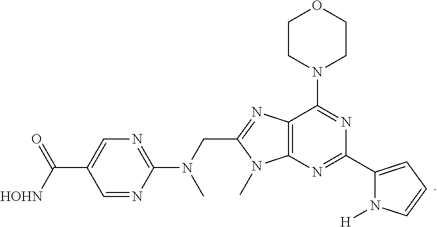

### Example 32. Preparation of a Formulation According to the Present Invention

The formulation was prepared by reference to the preparation method of Example 5. The API was the mesylate of

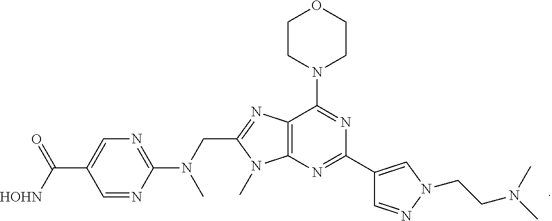

### Example 33. Preparation of a Formulation According to the Present Invention

The formulation was prepared by reference to the preparation method of Example 5. The API was the mesylate of

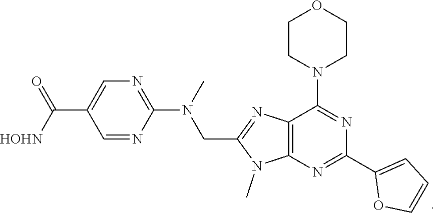

### Example 34. Preparation of a Formulation According to the Present Invention

The formulation was prepared by reference to the preparation method of Example 5. The API was the mesylate of

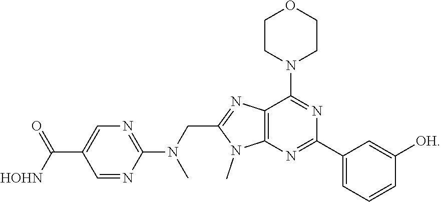

### Example 35. Preparation of a Formulation According to the Present Invention

The formulation was prepared by reference to the preparation method of Example 5. The API was the mesylate of

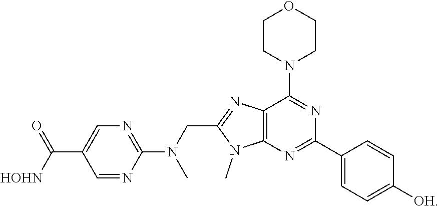

### Example 36. Preparation of a Formulation According to the Present Invention

The formulation was prepared by reference to the preparation method of Example 5. The API was the mesylate of

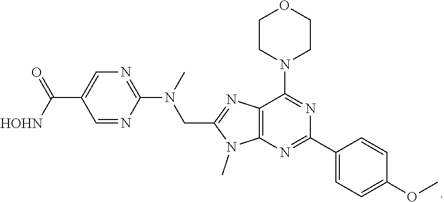

### Example 37. Preparation of a Formulation According to the Present Invention

The formulation was prepared by reference to the preparation method of Example 5. The API was the mesylate of

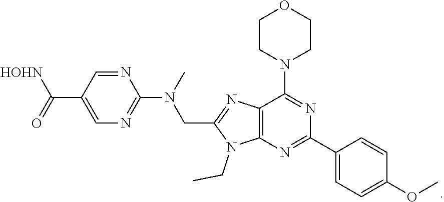

### Example 38. Preparation of a Formulation According to the Present Invention

The formulation was prepared by reference to the preparation method of Example 5. The API was the mesylate of

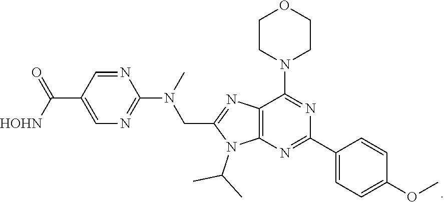

### Example 39. Preparation of a Formulation According to the Present Invention

The formulation was prepared by reference to the preparation method of Example 5. The API was the mesylate of

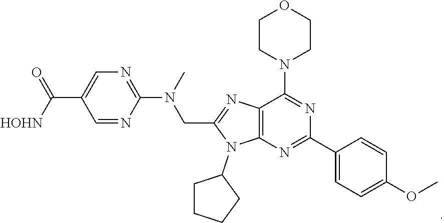

### Example 40. Preparation of a Formulation According to the Present Invention

The formulation was prepared by reference to the preparation method of Example 5. The API was the mesylate of

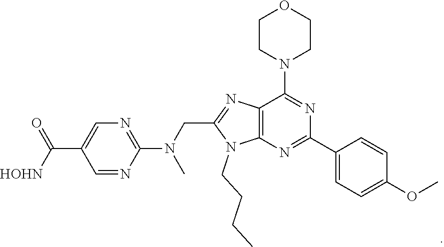

### Example 41. Preparation of a Formulation According to the Present Invention

The formulation was prepared by reference to the preparation method of Example 5. The API was the mesylate of

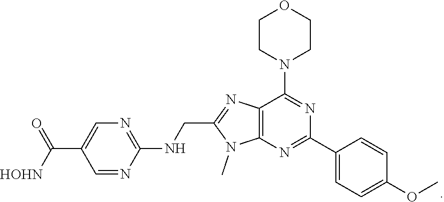

### Example 42. Preparation of a Formulation According to the Present Invention

The formulation was prepared by reference to the preparation method of Example 5. The API was the mesylate of

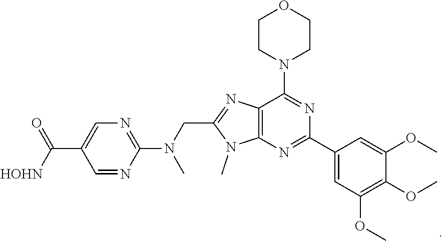

### Example 43. Preparation of a Formulation According to the Present Invention

The formulation was prepared by reference to the preparation method of Example 5. The API was the mesylate of

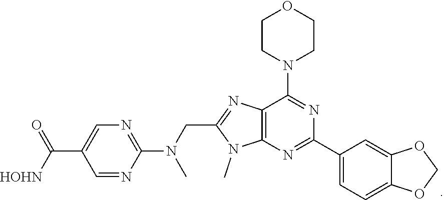

### Example 44. Preparation of a Formulation According to the Present Invention

The formulation was prepared by reference to the preparation method of Example 5. The API was the mesylate of

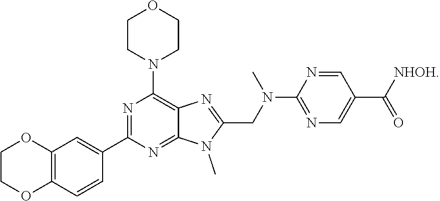

### Example 45. Preparation of a Formulation According to the Present Invention

The formulation was prepared by reference to the preparation method of Example 5. The API was the mesylate of

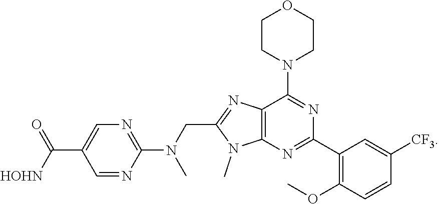

### Example 46. Preparation of a Formulation According to the Present Invention

The formulation was prepared by reference to the preparation method of Example 5. The API was the mesylate of

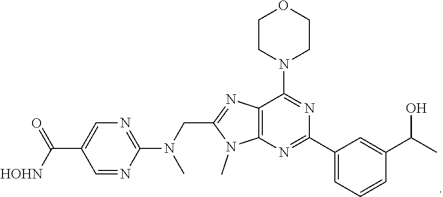

### Example 47. Preparation of a Formulation According to the Present Invention

The formulation was prepared by reference to the preparation method of Example 5. The API was the mesylate of

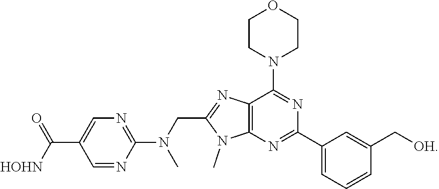

### Example 48. Preparation of a Formulation According to the Present Invention

The formulation was prepared by reference to the preparation method of Example 5. The API was the mesylate of

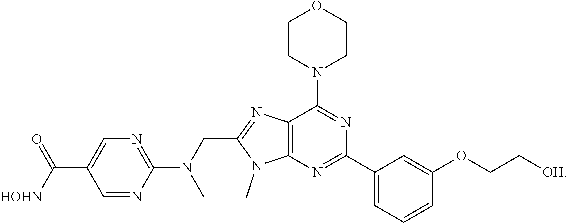

### Example 49. Preparation of a Formulation According to the Present Invention

The formulation was prepared by reference to the preparation method of Example 5. The API was the mesylate of

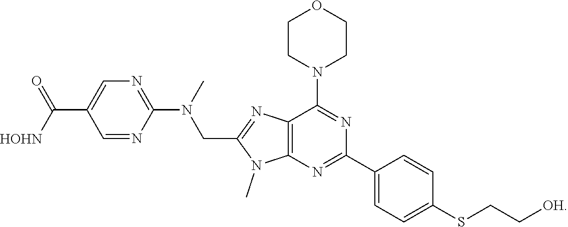

### Example 50. Preparation of a Formulation According to the Present Invention

The formulation was prepared by reference to the preparation method of Example 5. The API was the mesylate of

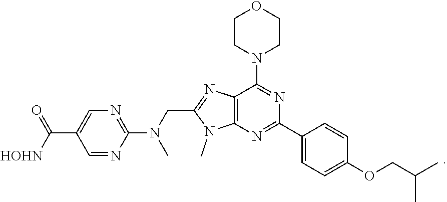

### Example 51. Preparation of a Formulation According to the Present Invention

The formulation was prepared by reference to the preparation method of Example 5. The API was the mesylate of

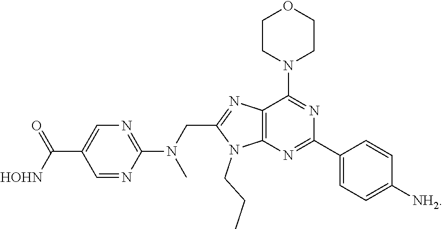

### Example 52. Preparation of a Formulation According to the Present Invention

The formulation was prepared by reference to the preparation method of Example 5. The API was the mesylate of

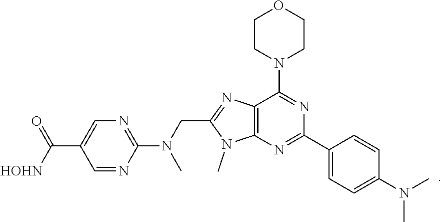

### Example 53. Preparation of a Formulation According to the Present Invention

The formulation was prepared by reference to the preparation method of Example 5. The API was the mesylate of

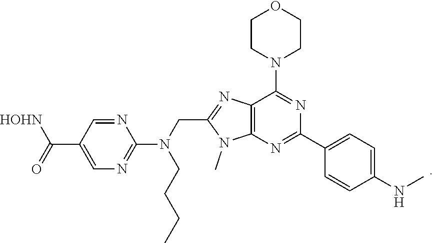

### Example 54. Preparation of a Formulation According to the Present Invention

The formulation was prepared by reference to the preparation method of Example 5. The API was the mesylate of

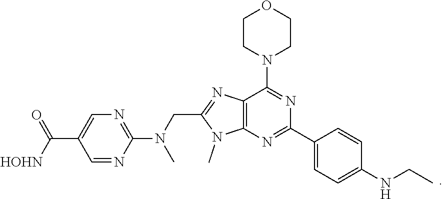

### Example 55. Preparation of a Formulation According to the Present Invention

The formulation was prepared by reference to the preparation method of Example 5. The API was the mesylate of

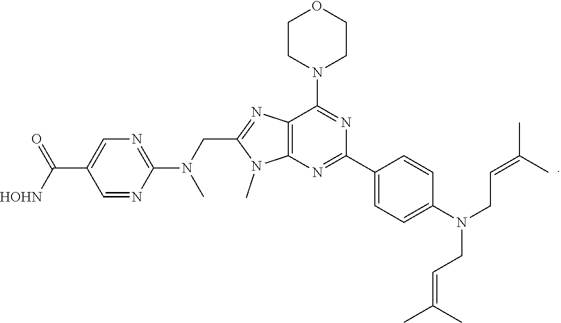

### Example 56. Preparation of a Formulation According to the Present Invention

The formulation was prepared by reference to the preparation method of Example 5. The API was the mesylate of

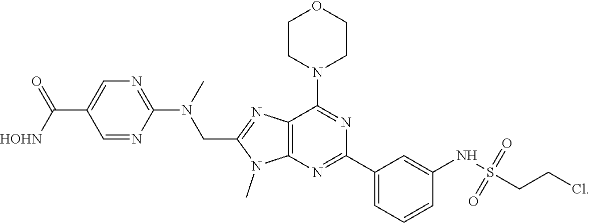

### Example 57. Preparation of a Formulation According to the Present Invention

The formulation was prepared by reference to the preparation method of Example 5. The API was the mesylate of

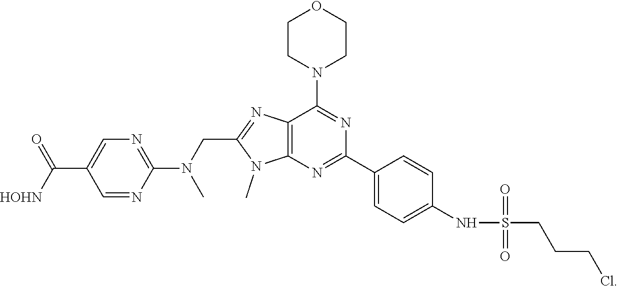

### Example 58. Preparation of a Formulation According to the Present Invention

The formulation was prepared by reference to the preparation method of Example 5. The API was the mesylate of

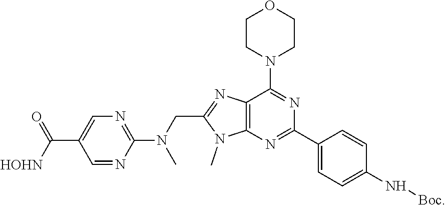

### Example 59. Preparation of a Formulation According to the Present Invention

The formulation was prepared by reference to the preparation method of Example 5. The API was the mesylate of

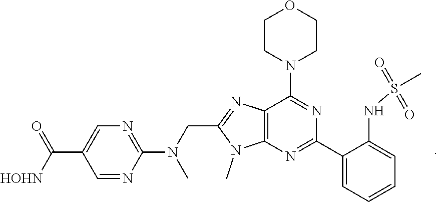

### Example 60. Preparation of a Formulation According to the Present Invention

The formulation was prepared by reference to the preparation method of Example 5. The API was the mesylate of

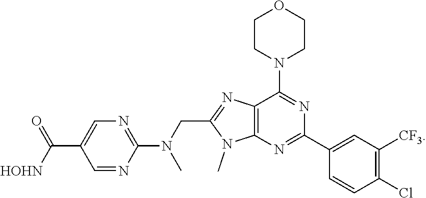

### Example 61. Preparation of a Formulation According to the Present Invention

The formulation was prepared by reference to the preparation method of Example 5. The API was the mesylate of

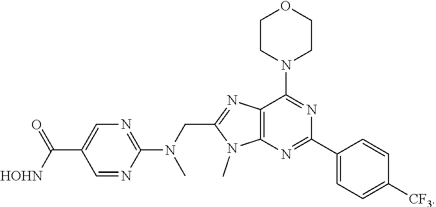

### Example 62. Preparation of a Formulation According to the Present Invention

The formulation was prepared by reference to the preparation method of Example 5. The API was the mesylate of

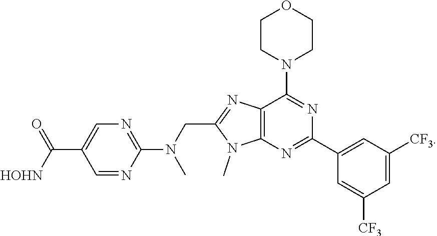

### Example 63. Preparation of a Formulation According to the Present Invention

The formulation was prepared by reference to the preparation method of Example 5. The API was the mesylate of

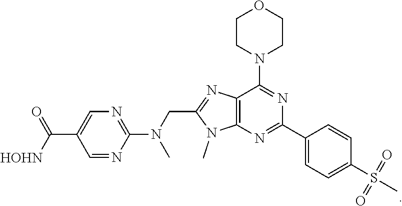

### Example 64. Preparation of a Formulation According to the Present Invention

The formulation was prepared by reference to the preparation method of Example 5. The API was the mesylate of

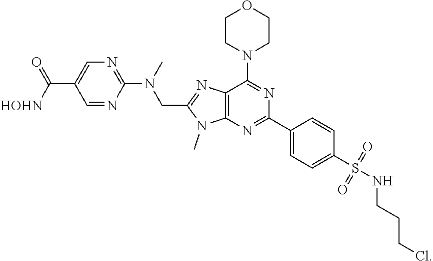

### Example 65. Preparation of a Formulation According to the Present Invention

The formulation was prepared by reference to the preparation method of Example 5. The API was the mesylate of

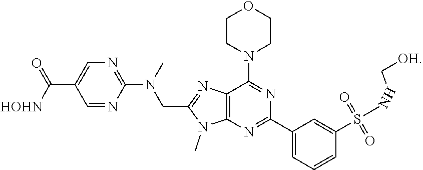

### Example 66. Preparation of a Formulation According to the Present Invention

The formulation was prepared by reference to the preparation method of Example 5. The API was the mesylate of

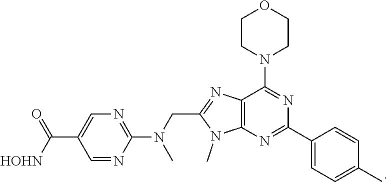

### Example 67. Preparation of a Formulation According to the Present Invention

The formulation was prepared by reference to the preparation method of Example 5. The API was the mesylate of

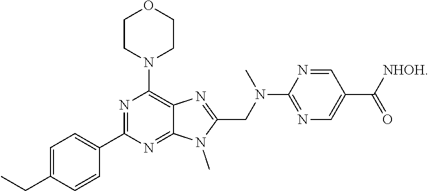

### Example 68. Preparation of a Formulation According to the Present Invention

The formulation was prepared by reference to the preparation method of Example 5. The API was the mesylate of

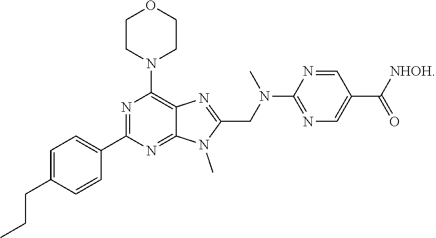

### Example 69. Preparation of a Formulation According to the Present Invention

The formulation was prepared by reference to the preparation method of Example 5. The API was the mesylate of

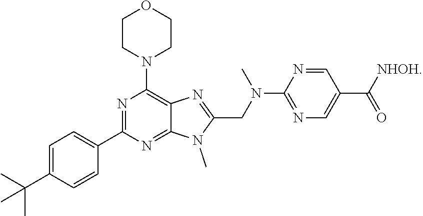

### Example 70. Preparation of a Formulation According to the Present Invention

The formulation was prepared by reference to the preparation method of Example 5. The API was the mesylate of

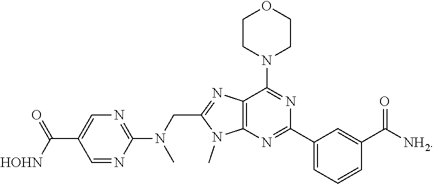

### Example 71. Preparation of a Formulation According to the Present Invention

The formulation was prepared by reference to the preparation method of Example 5. The API was the mesylate of

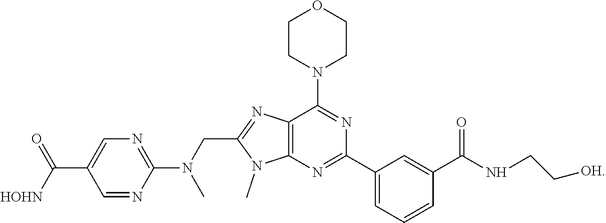

### Example 72. Preparation of a Formulation According to the Present Invention

The formulation was prepared by reference to the preparation method of Example 5. The API was the mesylate of

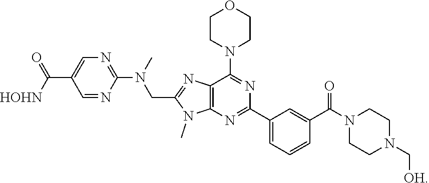

### Example 73. Preparation of a Formulation According to the Present Invention

The formulation was prepared by reference to the preparation method of Example 5. The API was the mesylate of

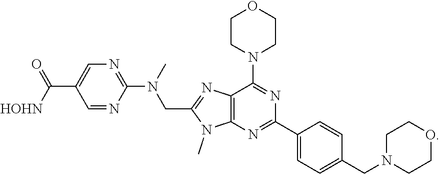

### Example 74. Preparation of a Formulation According to the Present Invention

The formulation was prepared by reference to the preparation method of Example 5. The API was the mesylate of

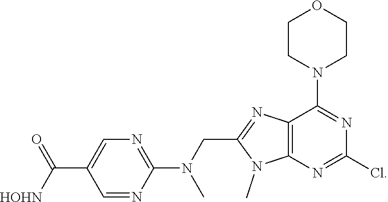

### Example 75. Preparation of a Formulation According to the Present Invention

The formulation was prepared by reference to the preparation method of Example 5. The API

was the mesylate of

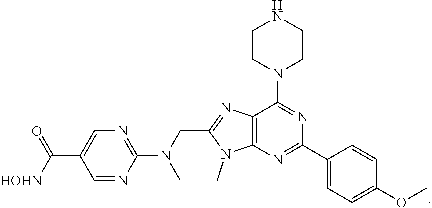

### Example 76. Preparation of a Formulation According to the Present Invention

The formulation was prepared by reference to the preparation method of Example 5. The API was the mesylate of

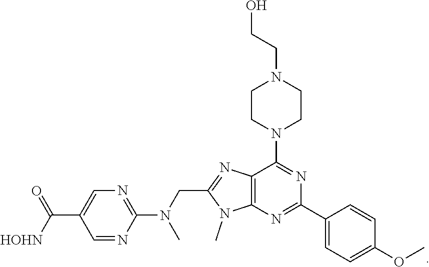

### Experiment Example 1: Screening of Excipients for the Preparation of the Present Invention

1. Selection of in situ basic salt-forming agents: Due to the extremely unstable hydroxamic acid of the API purinostat mesylate (PM), the in situ basic salt-forming agent was considered being used in the formulation to improve the stability. Common in situ basic salt-forming agents include arginine, lysine, and meglumine, which required further research. The solutions of arginine, lysine, and meglumine were respectively prepared, and then PM was added to saturation. The purity (%) and concentration (mg/mL) of PM were determined by high-performance liquid chromatography (HPLC), as well as the solubilization effects of three in situ basic salt-forming agents on PM and their impact on the purity of the PM main peak were studied. After being placed in a dark refrigerator at 4° C. for one day, whether there is any drug precipitation was observed using a clarity tester. The optimal sample was used for pH measurement.

TABLE 1
Selection of in situ basic salt-forming agents.

| | Solution 1 | Solution 2 | Solution 3 |
|---|---|---|---|
| | (Arginine) | (Lysine) | (Meglumine) |
| Arginine | 10% (M/V) | — | — |
| Lysine | — | Saturated | — |
| Meglumine | — | — | 10% (M/V) |
| API | Saturated | Saturated | Saturated |
| pH | 10.65 | — | 10.95 |
| Purity of API | 97.93% | 37.25 | 98.25 |
| Concentration | 1.54 mg/mL | 0.07 mg/mL | 2.07 mg/mL |
| of API | | | |
| Storing at | Trace | Slight | Trace |
| 4° C. for 1 d | sediment | sediment | sediment |

Using arginine or meglumine as an in situ basic salt-forming agent had a good solubilizing effect on PM, but still exhibited certain instability, and thus needed further optimization.

2. Selection of solubilizers: Cyclodextrin was widely used as a common solubilizer in pharmaceutics. The solutions of hydroxypropyl-β-cyclodextrin (HP-β-CD), sulfobutyl-β-cyclodextrin (SBE-β-CD), and β-cyclodextrin (β-CD) were respectively prepared, to which was then added PM to saturation. The purity and concentration of PM were determined by HPLC, to obtain the solubilization effects of three cyclodextrins on PM and their impact on the purity of PM main peak. The optimal sample was used for pH measurement. After being placed in a dark refrigerator at 4° C. for one day, whether there is any drug precipitation was observed using a clarity tester.

TABLE 2
Selection of cyclodextrin types.

| | Solution 4 | Solution 5 | Solution 6 |
|---|---|---|---|
| | (HP-β-CD) | (SBE-β-CD) | (β-CD) |
| HP-β-CD | 10% (M/V) | — | — |
| SBE-β-CD | — | 10% | — |
| β-CD | — | — | Saturated |
| | | | (1.85%) |
| API | Saturated | Saturated | Saturated |
| pH | 2.50 | 3.74 | 4.15 |
| Purity of API | 99.32% | 99.16% | 100% |
| Concentration | 2.89 mg/mL | 0.95 mg/mL | 0.42 mg/mL |
| of API | | | |
| Storing at | Trace | Trace | Slight |
| 4° C. for 1 d | sediment | sediment | sediment |

Compared with SBE-β-CD and β-CD, HIP-β-CD had a significantly better solubilizing effect on PM, but still exhibited certain instability, and required further optimization.

3. Phase solubility of the in-situ basic salt-forming agent and solubilizer

According to the pH guidance standard for injection, the optimal pH range for injection was 4-9. Due to the fact that the API was a strong acid and weak base salt, and the solution itself had a certain acidity, it was found that two cyclodextrins had little effect on the pH of the solution, while arginine and meglumine had a greater impact on the pH of the solution. Therefore, the concentrations of arginine and meglumine (M/V) were designed to be 1‰, 3‰, 5‰, 8‰, and 10‰; and the concentrations (M/V) of hydroxypropyl-β-cyclodextrin (HP-β-CD) and sulfobutyl ether-β-cyclodextrin (SBE-CD) were 5%, 10%, 15%, 20%, and 25%, respectively; then, the phase solubility tests were carried out.

3.1 Phase solubility data of cyclodextrins: A certain amount of swollen hydroxypropyl-β-cyclodextrin (HP-β-CD) solution (0.5 mg/mL) or sulfobutyl ether-β-cyclodextrin (SBE-CD) solution (0.5 mg/mL) was separately diluted with UP water to the volume, and prepared into solutions with concentrations (M/V) of 5%, 10%, 15%, 20%, and 25%, respectively. Then, excess PM raw materials were added and dissolved under ultrasound until saturation. A clear saturated solution was filtered out over a 0.22 μm membrane and transferred into a centrifuge tube. The concentration (mg/mL) and purity (%) of dissolved PM raw materials were detected by HPLC, and then, whether there was drug precipitation was observed using a clarity detector after being stored in a refrigerator at 4° C. in dark for one day. The relevant data are as follows:

TABLE 3
Phase solubility in HP-β-CD.

| HP-β-CD | 5% | 10% | 15% | 20% | 25% |
|---|---|---|---|---|---|
| Transferred amount | 600 μL | 1200 μL | 1800 μL | 2400 μL | 3000 μL |
| Dilution to | 6 mL | | | | |
| API | Saturated | | | | |
| pH values | 2.42 | 2.54 | 2.55 | 2.67 | 2.7 |
| Drug concentration | 1.85 | 2.02 | 3.16 | 4.13 | 4.52 |
| (mg/mL) | | | | | |
| Drug purity (%) | 99.21 | 99.15 | 99.08 | 98.96 | 98.24 |
| Storing at 4° C. | Trace | Trace | Trace | Trace | Trace |
| for one day | sedi- | sedi- | sedi- | sedi- | sedi- |
| | ment | ment | ment | ment | ment |

TABLE 4
Phase solubility in SBE-β-CD.

| SBE-β-CD | 5% | 10% | 15% | 20% | 25% |
|---|---|---|---|---|---|
| Transferred amount | 600 μL | 1200 μL | 1800 μL | 2400 μL | 3000 μL |
| Dilution to | 6 mL | | | | |
| API | Saturated | | | | |
| pH value | 3.77 | 3.71 | 3.67 | 3.64 | 3.57 |
| Drug concentration | 0.93 | 1.18 | 1.80 | 2.22 | 2.61 |
| (mg/mL) | | | | | |
| Drug purity (%) | 99.78 | 99.01 | 99.35 | 99.35 | 99.17 |
| Storing at 4° C. | Trace | Trace | Trace | Trace | Trace |
| for one day | sedi- | sedi- | sedi- | sedi- | sedi- |
| | ment | ment | ment | ment | ment |

Compared to SBE-β-CD, TIP-β-CD had a better solubilizing effect on PM at all test concentrations. The drug concentration was higher after the addition of HP-β-CD, so the solubilizing effect was better. But, as the dosage of HP-β-CD increased, it would affect the purity of PM, and so its dosage should be further optimized.

3.2 Phase solubility data of in-situ basic salt-forming agents: A certain amount of arginine (Arg) or meglumine was separately weighed and then diluted with UP water to the volume, so as to prepare into solutions with concentrations (M3V) of 1‰, 3‰, 5‰, 8‰, and 10‰, respectively. Then, excessive PM raw materials were added and dissolved under ultrasound until saturation. A clear saturated solution was filtered out over a 0.22 μm membrane and transferred into a centrifuge tube. The concentration (mg/mL) and purity (0%) of dissolved PM raw materials were detected by HPLC, and then, whether there was drug precipitation was observed using a clarity detector after being stored in a refrigerator at 4° C. in dark for one day. The relevant data are as follows:

TABLE 5
Phase solubility in arginine.

| Arginine | 1‰ | 3‰ | 5‰ | 8‰ | 10‰ |
|---|---|---|---|---|---|
| Weight | 10.10 mg | 30.20 mg | 50.10 mg | 80.40 mg | 101.10 mg |
| Dilution to | 6 mL | | | | |
| API | Saturated | | | | |
| pH value | 8.87 | 9.30 | 9.61 | 9.76 | 9.84 |
| Drug con- | 0.05 | 0.11 | 0.17 | 0.28 | 0.37 |
| centration | | | | | |
| (mg/mL) | | | | | |
| Drug purity | 100 | 100 | 100 | 98.97 | 99.24 |
| (%) | | | | | |

TABLE 6
Phase solubility in meglumine.

| Meglumine | 1‰ | 3‰ | 5‰ | 8‰ | 10‰ |
|---|---|---|---|---|---|
| Weight | 9.80 mg | 29.60 mg | 50.10 mg | 80.40 mg | 100.60 mg |
| Dilution to | 6 mL | | | | |
| API | Saturated | | | | |
| pH value | 9.65 | 9.98 | 10.22 | 10.41 | 10.42 |
| Drug con- | 0.13 | 0.24 | 0.35 | 0.46 | 0.70 |
| centration | | | | | |
| (mg/mL) | | | | | |
| Drug purity | 100 | 99.80 | 96.55 | 99.45 | 99.07 |
| (%) | | | | | |

The above results indicated that both arginine and meglumine could effectively solubilize PM.

Therefore, based on the above experimental results, hydroxypropyl-β-cyclodextrin (HP-j-CD), arginine, and meglumine were selected as excipients for PM of the present invention, and further amount screening of excipients was carried out as follows: An orthogonal experimental design was set up, and the corresponding amounts of raw materials and excipients were weighed according to the formula in Table 8, to which was respectively added 4 mL of water for injection. The formulations were prepared under conditions of different water bath temperatures, dissolution times, and mass ratios of raw material and excipients. The dissolution rate was observed with the naked eyes. After the dissolution time was over, the solution was made up to 6 mL with UP water, and then filtered over a 0.22 μm membrane to obtain a clear saturated solution, which was placed in a centrifuge tube. The concentration (mg/mL) and purity (%) of dissolved PM raw materials were detected using HPLC, and their pH values were measured with a pH meter. Then, the resultant solution was stored in a refrigerator at 4° C. in dark for one day, and whether there was any drug precipitation was determined using a clarity tester.

TABLE 8
Scale of factors influencing orthogonal experiments.

| | | | Time | Temperature |
|---|---|---|---|---|
| Levels | Arg/PM | HP-β-CD/PM | (h) | (° C.) |
| 1 | 2:1 | 10:1 | 0.5 | 30 |
| 2 | 3:1 | 15:1 | 1 | 40 |
| 3 | 4:1 | 20:1 | 2 | 50 |

TABLE 9
Orthogonal experimental table.

| | Time | | | Temperature |
|---|---|---|---|---|
| Formula | (h) | Arg/PM | HP-β-CD/PM | (° C.) |
| 1 | 0.5 | 2:1 | 10:1 | 30 |
| 2 | 0.5 | 3:1 | 20:1 | 40 |
| 3 | 0.5 | 4:1 | 15:1 | 50 |
| 4 | 1 | 2:1 | 15:1 | 40 |
| 5 | 1 | 3:1 | 10:1 | 50 |
| 6 | 1 | 4:1 | 20:1 | 30 |
| 7 | 2 | 2:1 | 20:1 | 50 |
| 8 | 2 | 3:1 | 15:1 | 30 |
| 9 | 2 | 4:1 | 10:1 | 40 |

TABLE 14
Experimental phenomena and results.

| Formula | 1 | 2 | 3 | 4 | 5 | 6 | 7 | 8 | 9 |
|---|---|---|---|---|---|---|---|---|---|
| Dissolution rate | Slow | Slow | Fast | Slow | Slow | Fast | Fast | Slow | Slow |
| End | Milky | Trace | Dissolved | Milky | Milky | Dissolved | Dissolved | Milky | Milky |
| | | precipitation | and clear | | | and clear | and clear | | |
| Dilution to | 6 mL | | | | | | | | |
| PM concentration | 2.57 | 4.98 | 4.96 | 4.16 | 3.57 | 4.72 | 4.77 | 4.41 | 4.26 |
| PM purity | 99.08 | 98.92 | 98.70 | 99.12 | 98.98 | 98.75 | 98.76 | 99.09 | 99.00 |
| pH value | 9.50 | 9.56 | 9.77 | 9.49 | 9.64 | 9.80 | 9.35 | 9.70 | 9.81 |
| Storing at 4° C. | — | Trace | Trace | — | — | Trace | Trace | — | — |
| for one day | | sediment | sediment | | | sediment | sediment | | |

In practical use, the injection formulation of PM needed to have a PM concentration of >2 mg/mL. From the results of the above orthogonal experiment, formulas 1-9 all met the concentration requirements. Among them, formulas 2, 3, 4, 6, 7, 8, and 9 had a API concentration of >4 mg/mL, and the drug purity was not obviously affected, that indicated a very good solubilization effect, and the drug was not easy to precipitate during dilution prior to administration. Therefore, when the mass ratio of HP-β-CD, Arg, and PM was (2-4):(10-20):1, a good solubilization effect could be realized. More further, formulas 3, 6, and 7 had the advantage of fast dissolution rate, and therefore the mass ratio of HP-β-CD, Arg, and PM being (2-4):(10-20):1 was more preferred; however, formulas 3, 6, and 7 still had stability issues, that is, there was trace precipitation after being stored at 4° C. for one day. Considering that in the screening of in situ basic salt-forming agents mentioned above, in addition to arginine having a good solubilizing effect on PM, meglumine also had a good solubilizing effect on PM, and thus it was proposed to add a suitable amount of meglumine to further improve the stability of the formulation.

In cases where the effects were comparable, a lower amount of excipients would be more advantageous. Therefore, based on the amount of cyclodextrin used, the above formula 3 was chosen as the basis for further screening the amount of meglumine.

The amount screening of meglumine was as follows:

According to the amount in the formula, the purinostat mesylate (PM) raw material and corresponding excipients (the amounts of HP-β-CD and Arg are shown in formula 3 of Table 14, while the amount of meglumine is shown in Table 15) were weighed, followed by addition of a certain amount of ultrapure water, and then the resultant solution was stirred at 50° C. under 900 rpm for 0.5 h to dissolve. The dissolution was observed with the naked eye, and then the solution was made up to the volume. The resultant solution was filtered over a 0.22 μm membrane to obtain a clear solution, which was then transferred into a transparent penicillin bottle. After being refrigerated for a certain period of time, its stability was observed under a clarity tester.

TABLE 1
The amount screening of meglumine.

| Formula No. | Formula A | Formula B | Formula C | Formula D | Formula E |
|---|---|---|---|---|---|
| Concentration of | 0.00% | 0.75% | 1.50% | 2.25% | 3.00% |
| meglumine | | | | | |
| Meglumine:PM | 0 | 1.5:1 | 3:1 | 4.5:1 | 6:1 |
| (mass ratio) | | | | | |
| Final dissolution | Dissolved | No | No | No | No |
| | and clear | precipitation | precipitation | precipitation | precipitation |
| Cold-storage for | Trace | No | No | No | No |
| one day | sediment | precipitation | precipitation | precipitation | precipitation |
| Cold-storage for | Trace | No | No | No | No |
| three days | sediment | precipitation | precipitation | precipitation | precipitation |
| Cold-storage for | Slight | Trace | Trace | Trace | Trace |
| seven days | sediment | sediment | sediment | sediment | sediment |

The above results indicated that adding a certain amount of meglumine could help to further improve the stability of the formulation solution. In cases where the effect was equivalent, a less amount of excipients would be more advantageous. The most preferred is formula B, which not only had good solubilization effect, but also good stability and less excipients. The preparation of the present invention was recommended to be used within one day of being left in solution.

In conclusion, the use of cyclodextrin, arginine, and meglumine was beneficial for increasing the solubility and stability of a HDACI purinostat mesylate. Especially when three excipients of cyclodextrin, arginine, and meglumine were used simultaneously, the solubility and stability of PM preparations were the highest. When the mass ratio of cyclodextrin, arginine, meglumine, and inhibitors was 15:4:1.5:1, not only the excellent solubility and stability of the injection could be ensured, but also the injection had a fast dissolution rate and a small amount of excipients, and thus this mass ratio was the most preferable.

### Experimental Example 2: Pharmacodynamic Experiment of the Preparation According to the Present Invention on Subcutaneously Transplanted Tumor of Human Burkitt's Lymphoma Daudi Cell Lines

### 1. Experimental Methods

Establishment of subcutaneously transplanted tumor model: Human Burkitt's lymphoma Daudi cell lines were in vitro cultured in RPMI-1640 medium containing 10% fetal bovine serum, 100 U/mL penicillin, and 100 μg/mL streptomycin. The cells in logarithmic growth phase were selected, and washed three times with PBS buffer under sterile conditions. The single cell suspension was diluted with PBS buffer to 2×10⁸ cells/mL for future use. Once seeded, 0.1 mL of single cell suspension was taken, and then subcutaneously inoculated into the back of female NOD/SCID mice with a weight of >20 g. Grouping and treatment: When the volume of the tumor reached 100-200 mm³ or above (the measurement and calculation method of tumor volume (TV): TV (mm³)=a×b2×π/6, wherein a and b represent the longest diameter and the shortest diameter (mm), respectively), tumor-bearing mice with excessive or insufficient tumor volumes would be eliminated, and qualified animals were randomly divided into groups. The grouping rules are shown in the table below:

| | Animal | | Injection | Drug | |
|---|---|---|---|---|---|
| | numbers | Dosage | volume | delivery | Administration |
| Groups | (mice) | (mg/kg) | (μL/g) | route | frequency |
| Control | 7 | — | 10 | i.v. | Three times |
| | | | | | a week |
| Vehicle | 7 | — | 10 | i.v. | Three times |
| | | | | | a week |
| CODOX | 7 | — | — | — | — |
| LBH589 | 7 | 10 | 10 | i.v. | Three times |
| | | | | | a week |
| Example 5 | 7 | 1.25 | 10 | i.v. | Three times |
| of the | | | | | a week |
| present | 7 | 2.5 | 10 | i.v. | Three times |
| invention | | | | | a week |
| | 7 | 5 | 10 | i.v. | Three times |
| | | | | | a week |
| | 7 | 10 | 10 | i.v. | Three times |
| | | | | | a week |

Among them, the experimental animals in the control group were injected with the same volume of physiological saline as the highest dose group treated with injectable PM; the experimental animals in the vehicle group were injected with the same volume of blank formulations as the highest dose group of injectable PM, in which the blank formulation did not contain PM, but contained the same excipients. The CODOX group details are shown in the table below:

| | | Injection | Drug | |
|---|---|---|---|---|
| | Dosage | volume | delivery | Administration frequency |
| Drug | (mg/kg) | (μL/g) | pathway | or schedule |
| Cyclophosphamide | 20 | 10 | i.p. | 21 days is a cycle, with a |
| | | | | single administration on the |
| | | | | first day of the cycle |
| Vincristine | 0.25 | 10 | i.v. | 21 days is a cycle, with a |
| sulfate | | | | single administration on the |
| | | | | first day of the cycle |
| Doxorubicin | 1.65 | 10 | i.v. | 21 days is a cycle, with a |
| hydrochloride | | | | single administration on the |
| | | | | first day of the cycle |
| Methotrexate | 10 | 10 | i.p. | 21 days is a cycle, with |
| | | | | administrations on days 2, |
| | | | | 5, 9, and 12. |
| Cytarabine | 75 (the | 10 | i.p. | 21 days is a cycle, with |
| | second batch) | | | administrations on days 3, |
| | | | | 6, 10, and 13. |

Experimental endpoint and analysis indicators: Example 5 of the present invention was administered 14 times, and the experiment was terminated on the 32nd day after the first treatment. The analysis indicators are:

(1) The relative tumor volume (RTV) was calculated based on the measurement of subcutaneous tumor volume using the following formula: RTV=Vt/V0, wherein V0 is the TV measured for each group when the animals were divided into cages for administration (i.e. d0), and Vt is the TV measured for each group.

The evaluation index for anti-tumor activity was the relative tumor growth rate T/C (%), and the calculation formula was as follows:

$$T / C ( \% ) = \frac{TRTV}{CRTV} \times 100 \%$$

TRTV: the relative tumor volume of the treatment group; CRTV: the relative tumor volume of the negative control group.

(2) Determination of tumor weight and calculation of tumor inhibition rate (%)

When the experiment was terminated, the animals was euthanized according to the operating procedures of animal experiments, and dissected to collect the tumor mass, which was then weighted. The tumor inhibition rate (%) was calculated according to the following formula:

$$The tumor inhibition rate ( \% ) = \frac{The mean tumor weight of negative control group ( g ) - The mean tumor weight of each treatment group ( g ) .}{The mean tumor weight of negative control group ( g ) .} \times 100 \% .$$

(3) Animal survival and weight changes during the experimental process.

### 2. Experimental Results

| | | Animal | Weight of tumor mass | |
|---|---|---|---|---|
| | | numbers | | Tumor |
| | Dosage | End/ | | inhibition |
| Grouping | mg/kg | Begining | X ± SD | rate (%) |
| Control | — | 7/7 | 3.58 ± 0.38 | — |
| Vehicle | — | 7/7 | 3.57 ± 0.61 | 0.16 |
| CODOX | — | 6/7 | 2.12 ± 0.42 | 40.83 |
| LBH589 | | 7/7 | 0.83 ± 0.38 | 76.92 |
| Example 5 of the | 1.25 | 7/7 | 1.09 ± 0.26 | 69.65 |
| present invention | 2.5 | 7/7 | 0.56 ± 0.18 | 84.38 |
| | 5 | 7/7 | 0.49 ± 0.24 | 86.18 |
| | 10 | 7/7 | 0.42 ± 0.12 | 88.14 |

According to the results, the preparation of the present invention had a good inhibitory rate on the tumor model, which was significantly better than other positive groups.

### Experimental Example 3: Pharmacodynamic Experiment of the Preparation According to the Present Invention on Subcutaneously Transplanted Tumor of Human Multiple Myeloma RPMI-8226 Cell Lines

### 1. Experimental Methods

Establishment of subcutaneously transplanted tumor model: Human multiple myeloma RPMI-8226 cell lines were in vitro cultured in RPMI-1640 medium containing 10% fetal bovine serum, 100 U/mL penicillin, and 100 μg/mL streptomycin. The cells in logarithmic growth phase were selected, and washed three times with PBS buffer under sterile conditions. The single cell suspension was diluted with PBS buffer to 7×10⁷ cells/mL for future use. Prior to seeding, 0.1 mL of single cell suspension was taken, and then subcutaneously inoculated into the back of female NOD/SCID mice with a weight of >20 g. Grouping and treatment: When the volume of the tumor reached 100-200 mm³ or above (the measurement and calculation method of tumor volume (TV): TV (mm³)=a×b2×π/6, wherein a and b represent the longest diameter and the shortest diameter (mm), respectively), tumor-bearing mice with excessive or insufficient tumor volumes would be eliminated, and qualified animals were randomly divided into groups. The grouping rules are shown in the table below:

| | | | Injection | Drug | |
|---|---|---|---|---|---|
| | Number | Dosage | volume | delivery | |
| Groups | (mice) | (mg/kg) | (μL/g) | pathway | Frequency |
| Control | 6 | — | 10 | i.v. | Three times |
| | | | | | a week |
| Vehicle | 6 | — | 10 | i.v. | Three times |
| | | | | | a week |
| LBH589 | 6 | 5 | 10 | i.v. | Three times |
| | | | | | a week |
| LBH589 + B + D | 6 | — | — | — | Three times |
| | | | | | a week |
| L + B + D | 6 | — | — | — | Three times |
| | | | | | a week- |
| Example 5 of the | 6 | 1.25 | 10 | i.v. | Three times |
| present invention | | | | | a week |
| | 6 | 2.5 | 10 | i.v. | Three times |
| | | | | | a week |
| | 6 | 5 | 10 | i.v. | Three times |
| | | | | | a week |
| | 6 | 10 | 10 | i.v. | Three times |
| | | | | | a week |

Among them, the experimental animals in the control group were injected with the same volume of physiological saline as the highest dose group of injectable PM; the experimental animals in the vehicle group were injected with the same volume of blank formulations as the highest dose group of injectable PM, in which the blank formulation did not contain PM, but contained the same excipients. “B” means Bortezomib; “D” means Dexamethasone; “L” means Lenalidomide. The administration details are shown in the table below:

| | | Injection | Drug | Administration |
|---|---|---|---|---|
| | Dosage | volue | delivery | frequency or |
| Drugs | (mg/kg) | (μL/g) | pathway | schedule |
| Bortezomib (B) | 0.1 | 5 | i.p. | Three times |
| | | | | a week |
| Lenalidomide (L) | 15 | 5 | i.p. | Three times |
| | | | | a week |
| Dexamethasone (D) | 1 | 5 | i.p. | Three times |
| | | | | a week |

Experimental endpoint and analysis indicators: Example 5 of the present invention was administered 14 times, and the experiment was terminated on the 32nd day after the first treatment. The analysis indicators are:

(1) The relative tumor volume (RTV) was calculated based on the measurement of subcutaneous tumor volume using the formula: RTV=Vt/V0, wherein V0 is the TV measured for each group when the animals were divided into cages for administration (i.e. d0), and Vt is the TV measured for each group.

The evaluation index for anti-tumor activity was the relative tumor growth rate T/C (%), and the calculation formula was as follows:

$$T / C ( \% ) = \frac{TRTV}{CRTV} \times 100 \%$$

TRTV: the relative tumor volume of the treatment group; CRTV: the relative tumor volume of the negative control group.

(2) Determination of tumor weight and calculation of tumor inhibition rate (%)

When the experiment was terminated, according to the operating procedures of animal experiments, the animals was euthanized and dissected to collect the tumor mass, which was then weighted. The tumor inhibition rate (%) was calculated according to the following formula:

$$The tumor inhibition rate ( \% ) = \frac{\begin{matrix}The mean tumor weight of negative control group ( g ) - \\ The mean tumor weight of each treatment group ( g )\end{matrix}}{The mean tumor weight of negative control group ( g )} \times 100 \%$$

(3) Animal survival and weight changes during the experimental process.

### 2. Experimental Results

| | | Animal | Tumor weight (g) | |
|---|---|---|---|---|
| | | number | | Tumor |
| | Dosage | End/ | | inhibition |
| Groups | mg/kg | Beginning | X ± SD | rate (%) |
| Control | — | 6/6 | 2.24 ± 0.29 | — |
| Vehicle | — | 6/6 | 2.57 ± 0.69 | −14.97 |
| LBH589 | 10 | 6/6 | 0.90 ± 0.37 | 59.79 |
| LBH589 + B + D | — | 5/6 | 0.28 ± 0.08 | 87.49 |
| L + B + D | — | 4/6 | 0.24 ± 0.14 | 89.39 |
| Example 5 of the | 1.25 | 6/6 | 0.41 ± 0.24 | 81.61 |
| present invention | 2.5 | 6/6 | 0.17 ± 0.07 | 92.33 |
| | 5 | 6/6 | 0.05 ± 0.02 | 97.77 |
| | 10 | 6/6 | 0.05 ± 0.02 | 97.77 |

### Experimental Example 4: Pharmacodynamic Experiment of the Preparation According to the Present Invention on Subcutaneously Transplanted Tumor of Human Burkitt's Lymphoma Raji Cell Lines

### 1. Experimental Methods

Establishment of subcutaneously transplanted tumor model: Human Burkitt's lymphoma Raji cell lines were in vitro cultured in RPMI-1640 medium containing 10% fetal bovine serum, 100 U/mL penicillin, and 100 μg/mL streptomycin. The cells in logarithmic growth phase were selected, and washed three times with PBS buffer under sterile conditions. The single cell suspension was diluted with PBS buffer to 2×10⁸ cells/mL for future use. Prior to seeding, 0.1 mL of single cell suspension was taken out, and then subcutaneously inoculated into the back of female NOD/SCID mice with a weight of >20 g. Grouping and treatment was same as that of Experimental example 1.

### 2. Results

As shown in FIG. 4, the efficacy of the preparation according to the present invention was far superior to that of the positive group. The preparation of HDACIs prepared by the method of the present invention effectively retained the excellent anti-tumor activity of HDACIs.

In summary, the present invention provided a preparation of an HDACI, which significantly increased the solubility of HDACIs in water, improved the stability of HDACIs, retained the excellent anti-tumor activity of HDACIs, and broadened the application of HDACIs, suggesting an extremely high values in clinical practice and market.

1. A pharmaceutical composition, characterized in that it comprises a histone deacetylase inhibitor (HDACI) and an excipient;
The histone deacetylase inhibitor is a compound disclosed in the Chinese patent publication number CN107849045B, or a pharmaceutically acceptable salt, a solvate, an amide, an ester, an ether, a chemically protected form, and a prodrug thereof;
The excipient is at least one of or a combination comprising two or more of cyclodextrin, arginine, and meglumine.

2. The pharmaceutical composition according to claim 1, characterized in that the histone deacetylase inhibitor is a compound represented by formula I or a pharmaceutically acceptable salt, a solvate, an amide, an ester, an ether, a chemically protected form, and a prodrug thereof; 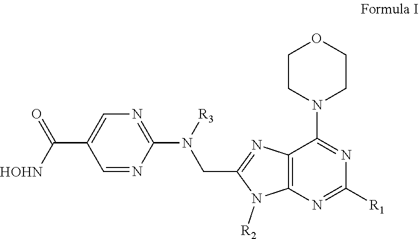
wherein, R₁ is selected from the group consisting of C₁-C₄ alkyl, C₁-C₄ alkoxy, —OH, halogen, C₃-C₈ cycloalkyl, —NH₂, 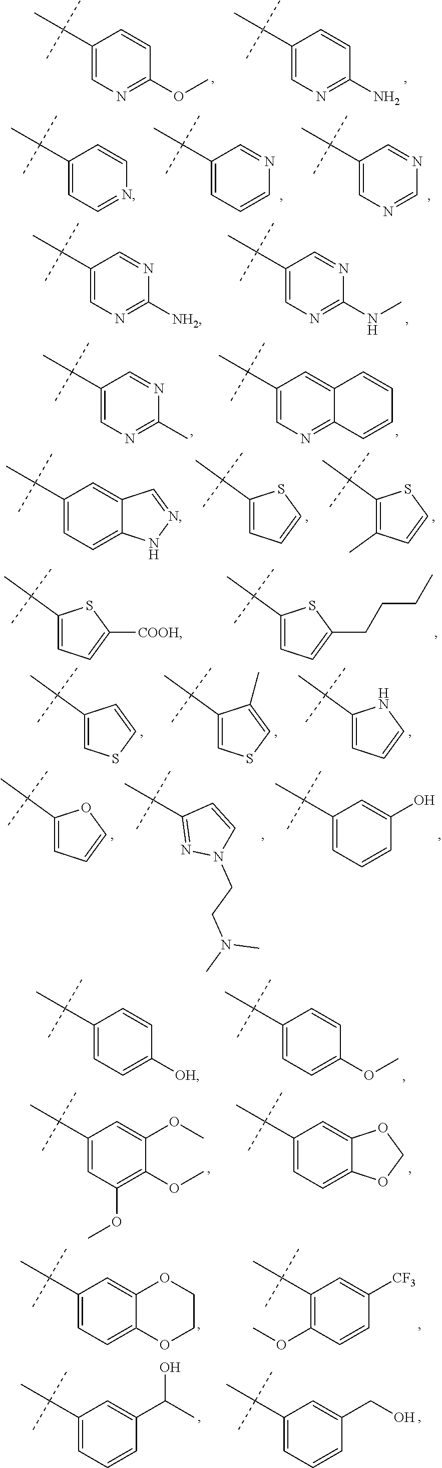 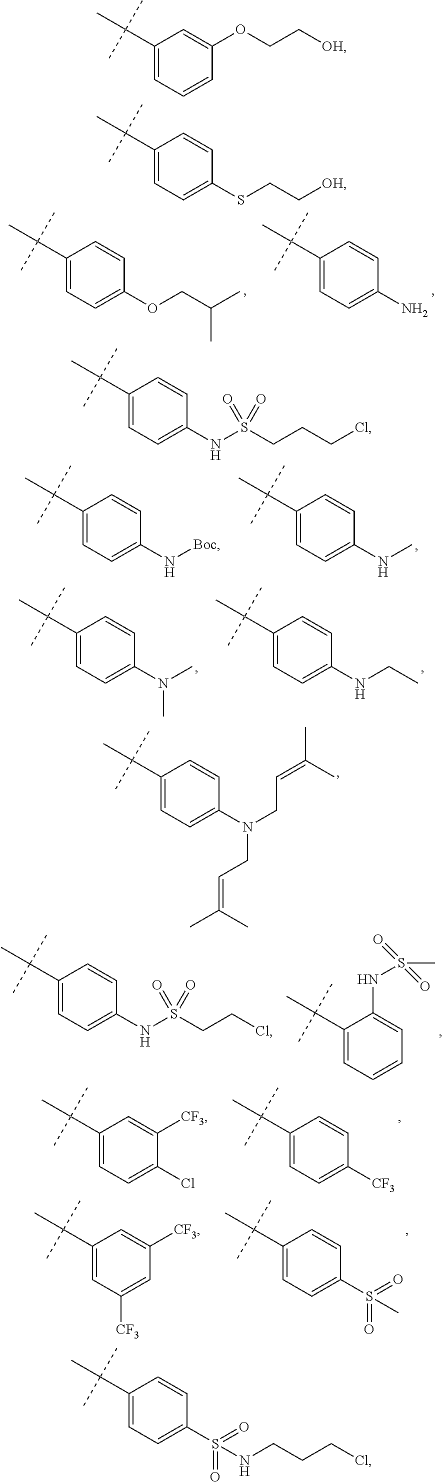 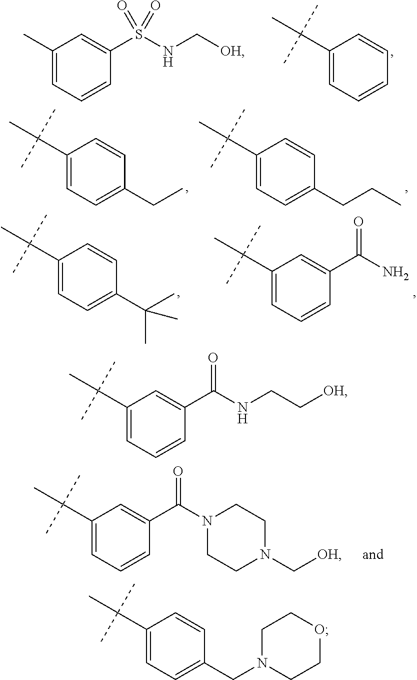
R₂ is selected from the group consisting of C₁-C₄ alkyl or C₃-C₈ cycloalkyl;
R₃ is selected from the group consisting of —H, C₁-C₄ alkyl, C₁-C₄ alkoxy, —OH, halogen or C₃-C₈ cycloalkyl;
the mass ratio of cyclodextrin to the inhibitor is (10-20):1; and/or the mass ratio of arginine to the inhibitor is (2-4):1; and/or the mass ratio of meglumine to the inhibitor is (1.5-6):1.

3. The pharmaceutical composition according to claim 2, characterized in that the compound represented by formula I is selected from the group consisting of:

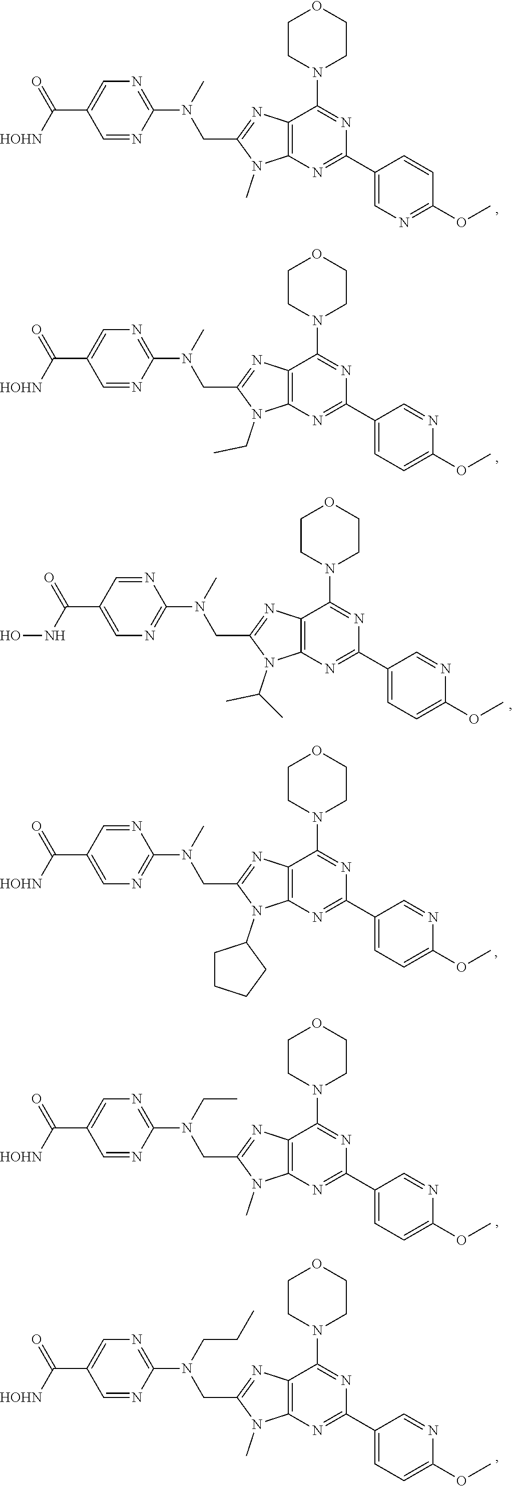

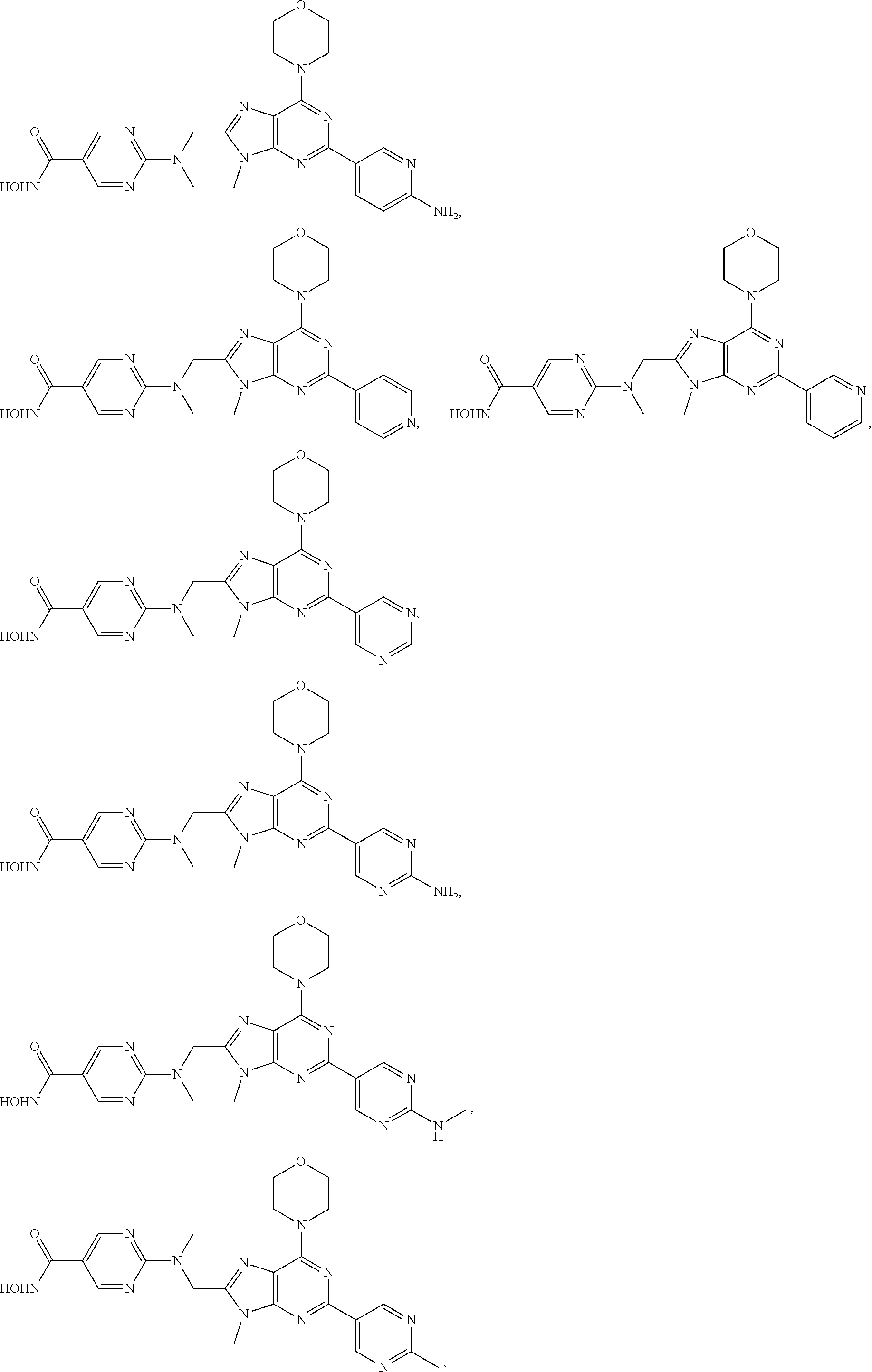

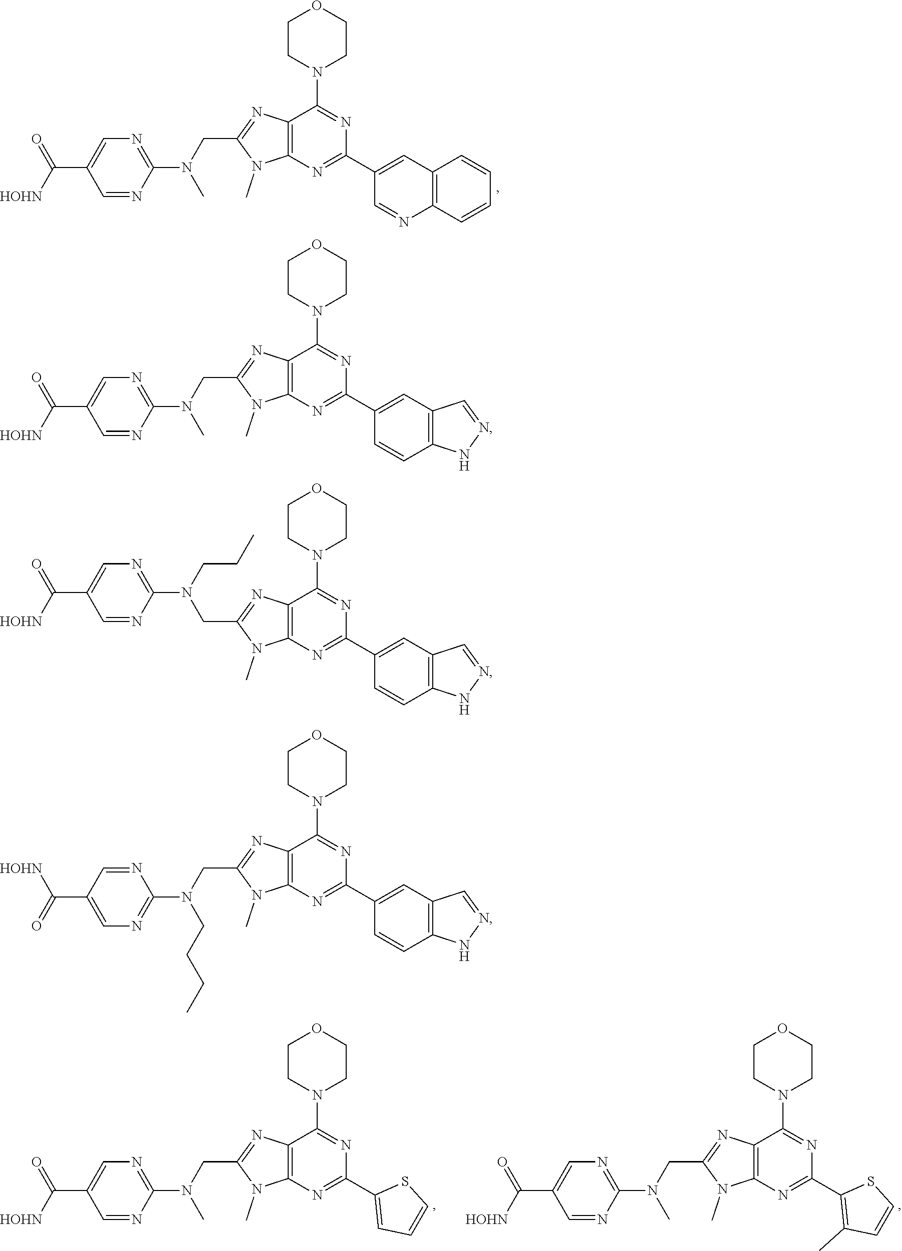

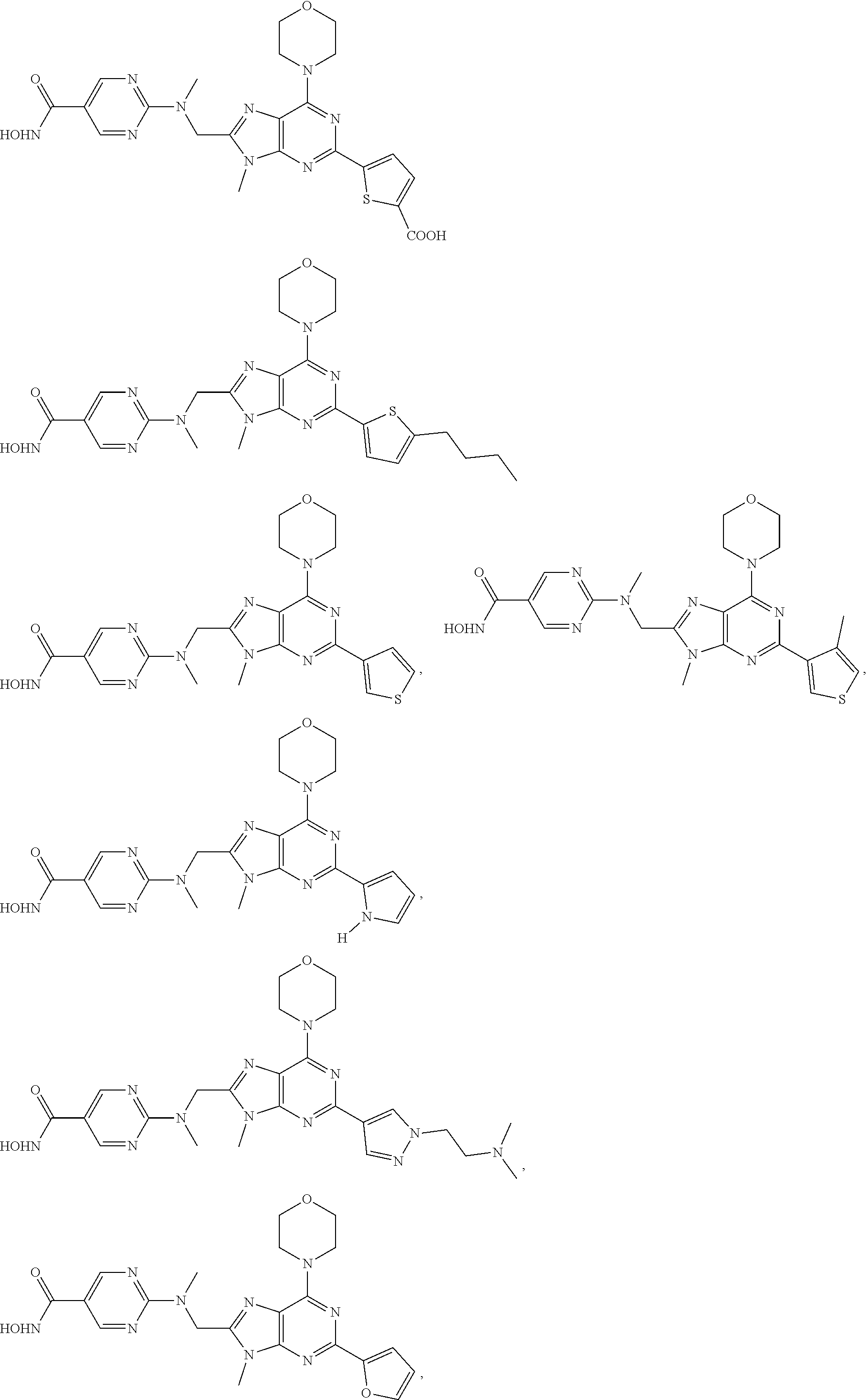

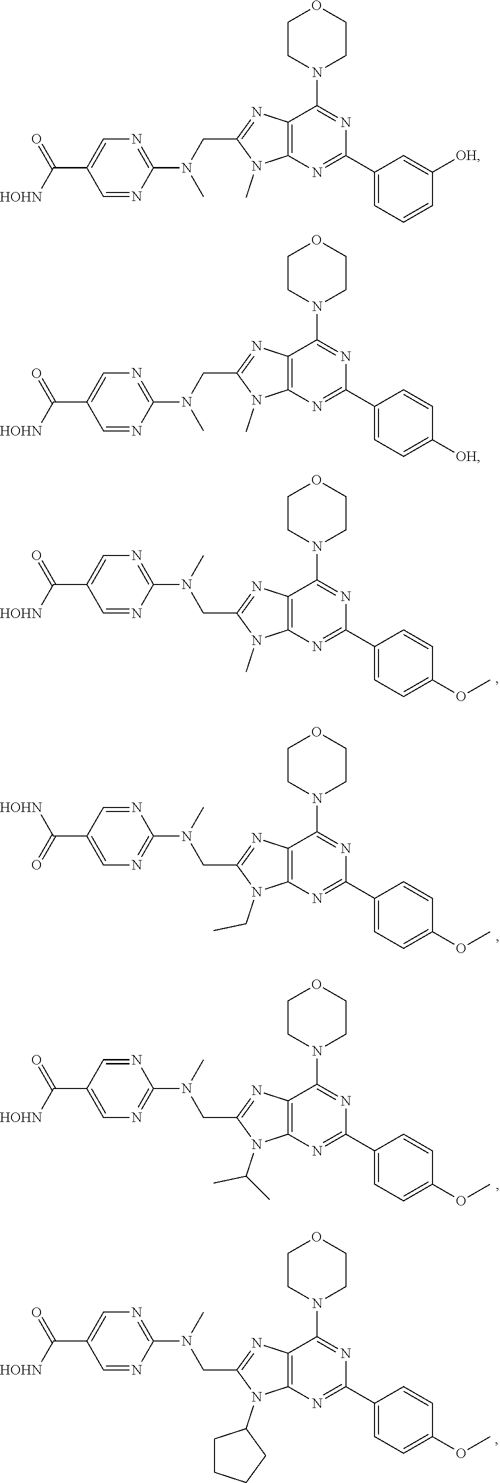

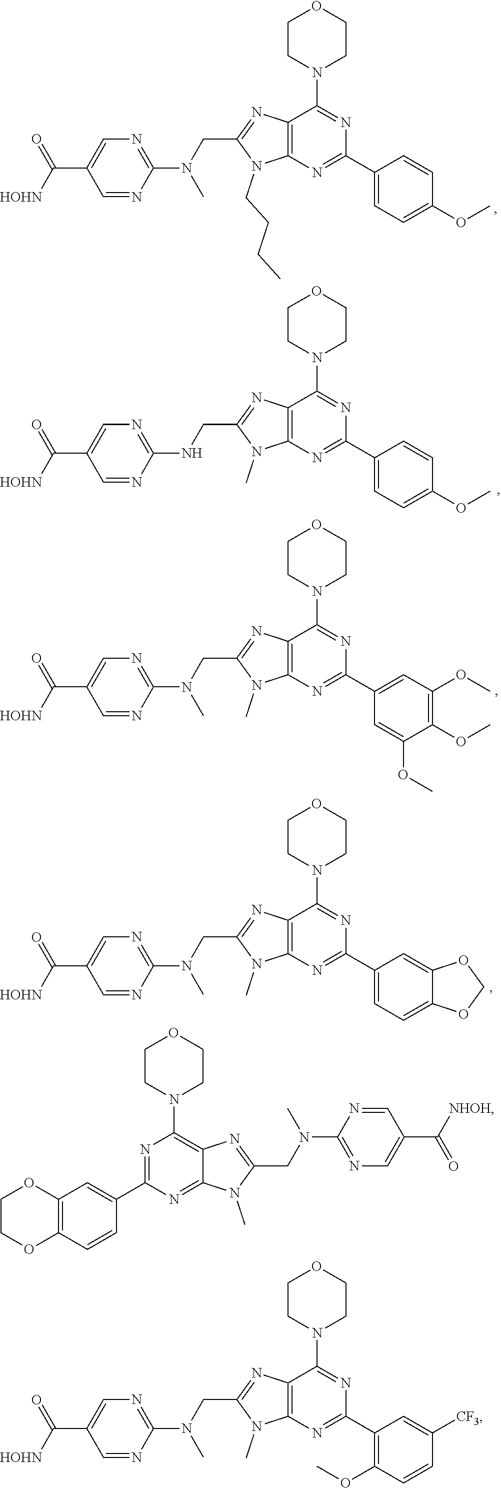

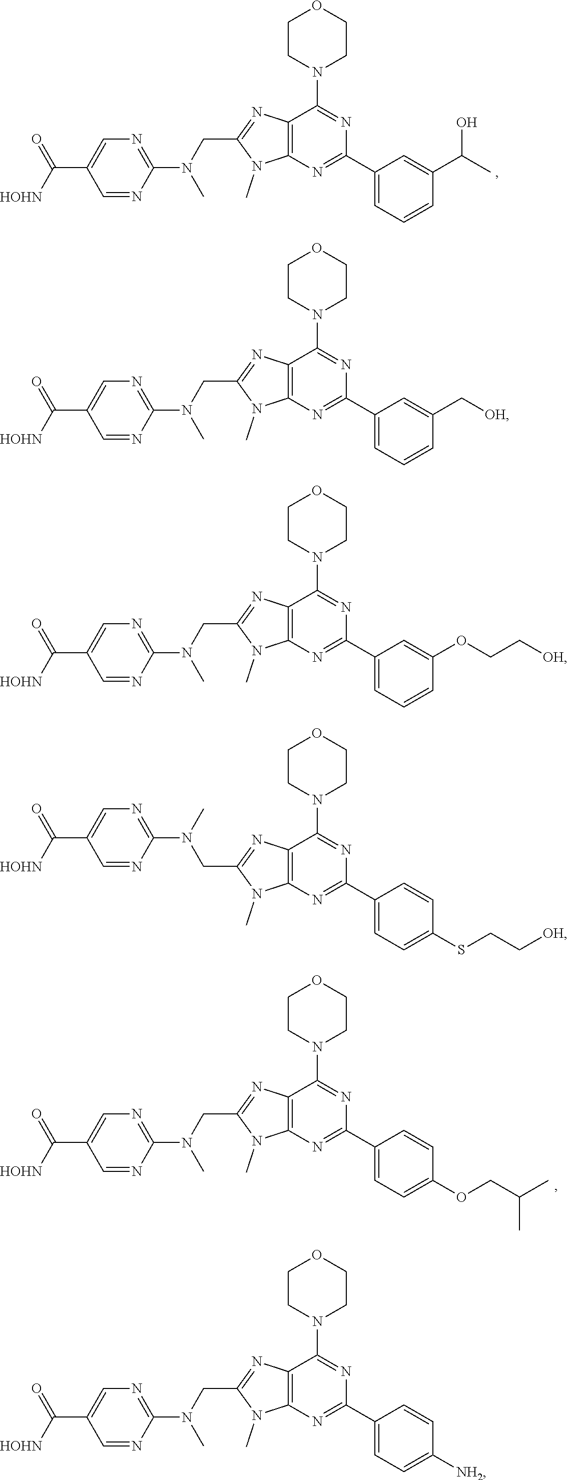

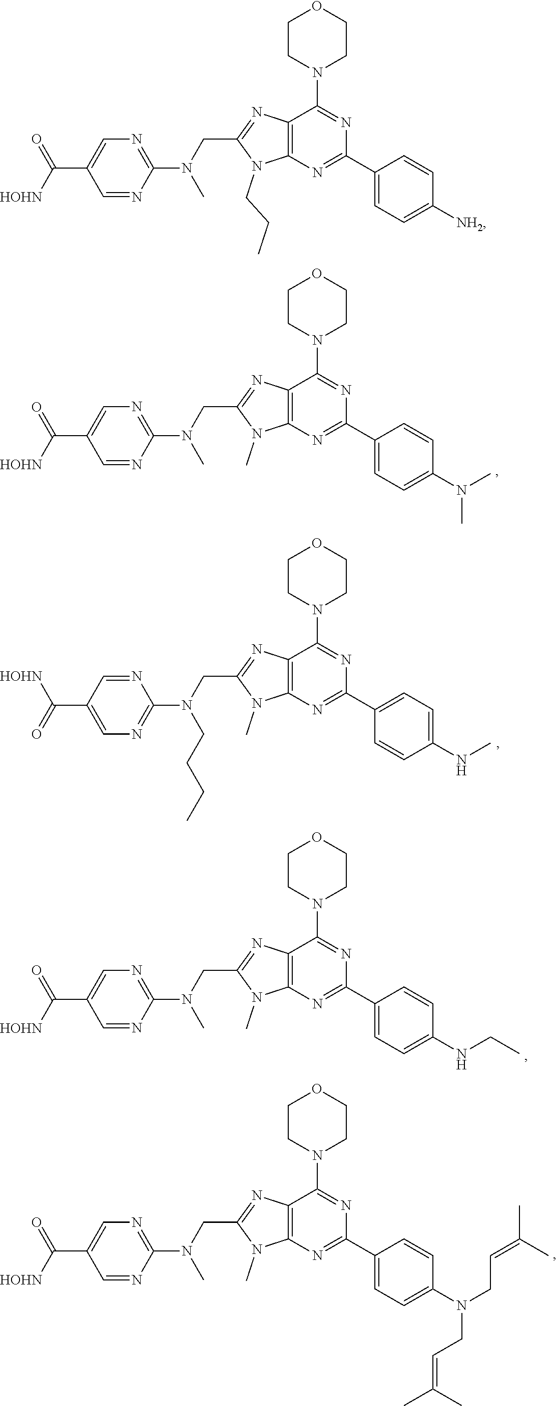

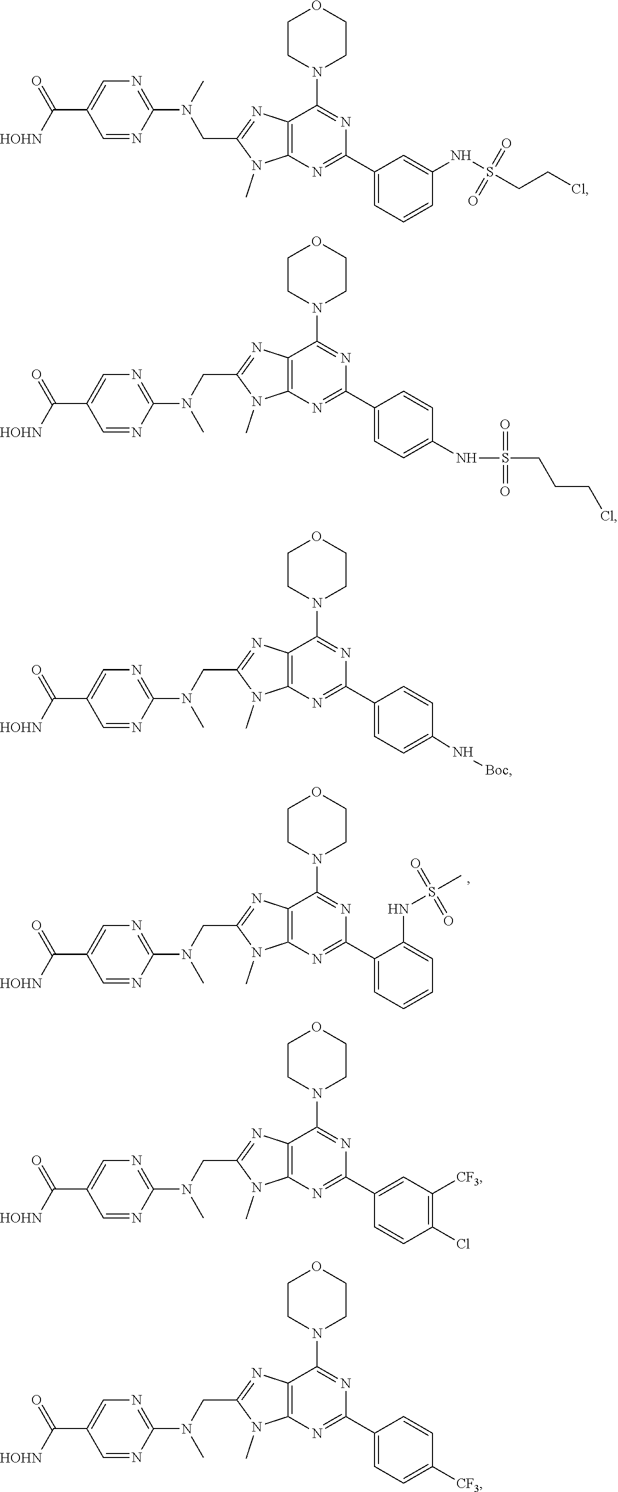

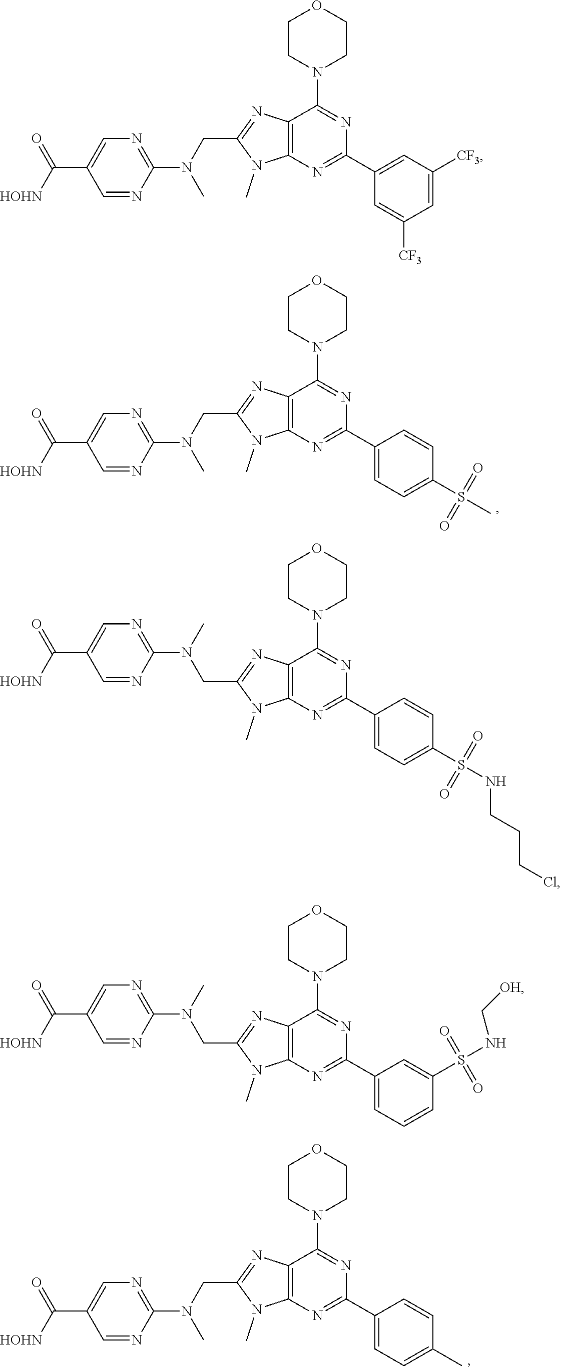

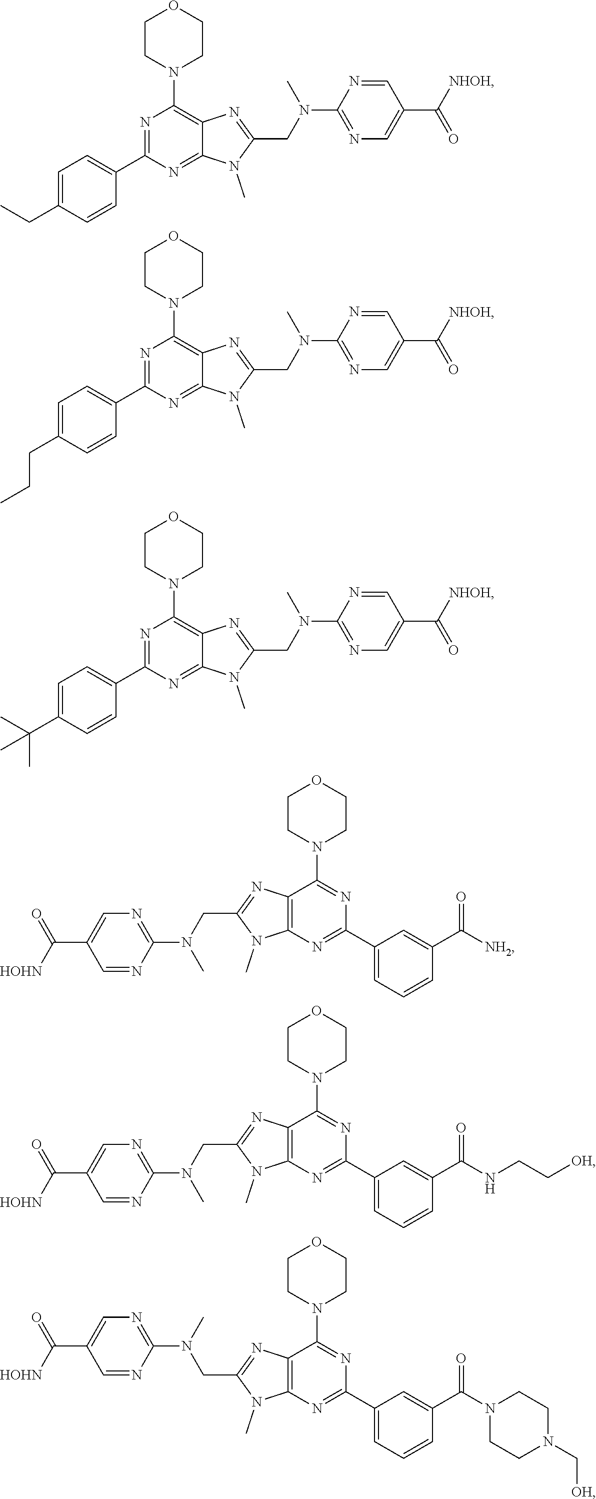

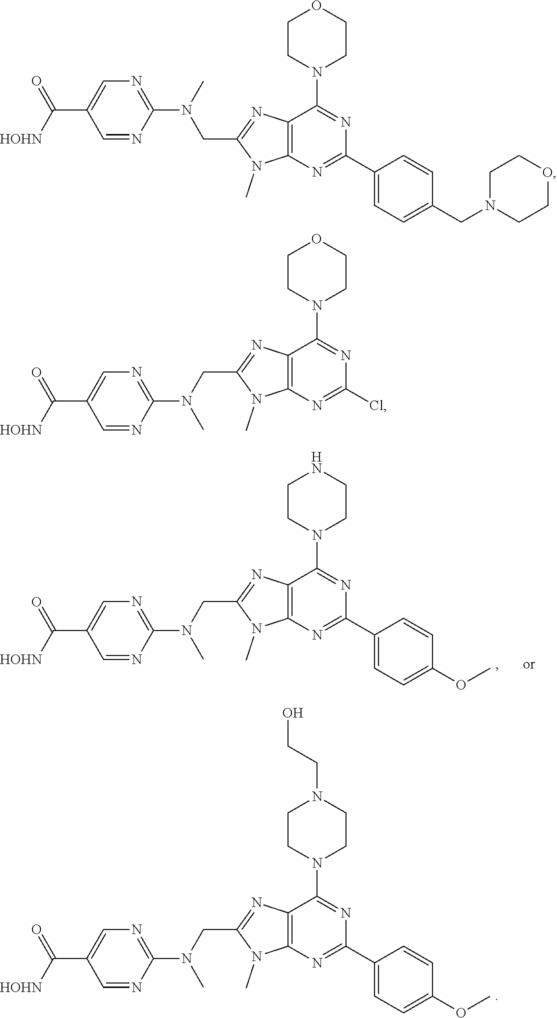

4. The pharmaceutical composition according to claim 2, characterized in that the excipient is selected from the group consisting of cyclodextrin, arginine, and meglumine, and the mass ratio of cyclodextrin to the inhibitor is (10-20):1; the mass ratio of arginine to the inhibitor is (2-4):1; the mass ratio of meglumine to the inhibitor is (1.5-6):1.

5. A pharmaceutical composition according to claim 1, characterized in that said cyclodextrin is selected from the group consisting of α-cyclodextrin, β-cyclodextrin, γ-cyclodextrin, (C1-4 alkyl)-α-cyclodextrin, (C1-4 alkyl)-β-cyclodextrin, (C1-4 alkyl)-γ-cyclodextrin, hydroxyl-(C1-4 alkyl)-α-cyclodextrin, hydroxyl-(C1-4 alkyl)-β-cyclodextrin, hydroxyl-(C1-4 alkyl)-β-cyclodextrin, carboxyl-(C1-4 alkyl)-α-cyclodextrin, carboxyl-(C1-4 alkyl)-β-cyclodextrin, carboxyl-(C1-4 alkyl)-γ-cyclodextrin, α-cyclodextrin ethers, β-cyclodextrin ethers, γ-cyclodextrin ethers, α-cyclodextrin sulfobutyl ether, β-cyclodextrin sulfobutyl ether, and γ-cyclodextrin sulfobutyl ether; preferably, said cyclodextrin is hydroxypropyl-β-cyclodextrin.

6. The pharmaceutical composition according to claim 1, characterized in that it is a preparation formed by the deacetylase inhibitor and excipients, in combination with pharmaceutically acceptable auxiliary ingredients.

7. The pharmaceutical composition according to claim 6, characterized in that the preparation is an injection, and the pharmaceutically acceptable auxiliary ingredients are water for injection, saline, glucose aqueous solution, saline for injection and infusion, glucose solution for injection and infusion, Ringer's solution, or Ringer's solution containing lactate.

8. The pharmaceutical composition according to claim 7, characterized in that the pharmaceutically acceptable auxiliary components are saline or glucose aqueous solution.

9. The pharmaceutical composition according to claim 6, characterized in that the concentration of histone deacetylase inhibitors in the formulation is 0.1-1000 mg/mL.

10. The preparation method of a pharmaceutical composition according to claim 6, characterized in that it comprises the following steps:
(1) The excipients are dissolved in the pharmaceutically acceptable auxiliary ingredients in a pre-determined ratio, to obtain the excipient solution;
(2) The histone deacetylase inhibitor is added to the excipient solution obtained in step (1), and then the resultant solution is stirred to dissolve, and filtered to obtain the pharmaceutical composition.
